SEQUENCE LISTING (182 sequences)

SEQ ID NO: 1 - RNA, 916 nt, Homo sapiens
ccagagaggggcaggcugguccccugacagguugaagcaaguagacgcccaggagccccgggagggggcugcaguuuccuuccuuccuucucggcagcgcuccgcgcccccaucgccccuccugcgcuagcggaggugaucgccgcggcgaugccggaggaggguucgggcugcucggugcggcgcaggcccuaugggugcguccugcgggcugcuuuggucccauuggucgcgggcuuggugaucugccucguggugugcauccagcgcuucgcacaggcucagcagcagcugccgcucgagucacuugggugggacguagcugagcugcagcugaaucacacaggaccucagcaggaccccaggcuauacuggcaggggggcccagcacugggccgcuccuuccugcauggaccagagcuggacaaggggcagcuacguauccaucgugauggcaucuacaugguacacauccaggugacgcuggccaucugcuccuccacgacggccuccaggcaccaccccaccacccuggccgugggaaucugcucucccgccucccguagcaucagccugcugcgucucagcuuccaccaagguuguaccauugccucccagcgccugacgccccuggcccgaggggacacacucugcaccaaccucacugggacacuuuugccuucccgaaacacugaugagaccuucuuuggagugcagugggugcgccccugaccacugcugcugauuaggguuuuuuaaauuuuauuuuauuuuauuuaaguucaagagaaaaaguguacacacaggggccacccgggguuggggugggaguguggugggggguagugguggcaggacaagagaaggcauugagcuuuuucuuucauuuuccuauuaaaaaauacaaaaaucaaaa

SEQ ID NO: 2 - RNA, 2794 nt, Homo sapiens
aggagccuacuuuauucugauaaaacaggucuaugcagcuaccaggacaauggaaucuacguugacuuuagcaacggaacaaccuguuaagaagaacacucuuaagaaauauaaaauagcuugcauuguucuucuugcuuugcuggugaucaugucacuuggauuaggccuggggcuuggacucaggaaacuggaaaagcaaggcagcugcaggaagaagugcuuugaugcaucauuuagaggacuggagaacugccggugugauguggcauguaaagaccgaggugauugcugcugggauuuugaagacaccuguguggaaucaacucgaauauggaugugcaauaaauuucguuguggagagaccagauuagaggccagccuuugcucuuguucagaugacuguuugcagaagaaagauugcugugcugacuauaagaguguuugccaaggagaaaccucauggcuggaagaaaacugugacacagcccagcagucucagugcccagaaggguuugaccugccaccaguuaucuuguuuucuauggauggauuuagagcugaauauuuauacacaugggauacuuuaaugccaaauaucaauaaacugaaaacauguggaauucauucaaaauacaugagagcuauguauccuaccaaaaccuucccaaaucauuacaccauugucacgggcuuguauccagagucacauggcaucauugacaauaauauguaugauguaaaucucaacaagaauuuuucacuuucuucaaaggaacaaaauaauccagccugguggcaugggcaaccaauguggcugacagcaauguaucaagguuuaaaagccgcuaccuacuuuuggcccggaucagaaguggcuauaaauggcuccuuuccuuccauauacaugccuuacaacggaagugucccauuugaagagaggauuucuacacuguuaaaauggcuggaccugcccaaagcugaaagacccagguuuuauaccauguauuuugaagaaccugauuccucuggacaugcagguggaccagucagugccagaguaauuaaagccuuacagguaguagaucaugcuuuugggauguugauggaaggccugaagcagcggaauuugcacaacugugucaauaucauccuucuggcugaccauggaauggaccagacuuauuguaacaagauggaauacaugacugauuauuuucccagaauaaacuucuucuacauguacgaagggccugccccccgcauccgagcucauaauauaccucaugacuuuuuuaguuuuaauucugaggaaauuguuagaaaccucaguugccgaaaaccugaucagcauuucaagcccuauuugacuccugauuugccaaagcgacugcacuaugccaagaacgucagaaucgacaaaguucaucucuuuguggaucaacaguggcuggcuguuaggaguaaaucaaauacaaauuguggaggaggcaaccaugguuauaacaaugaguuuaggagcauggaggcuaucuuucuggcacauggacccaguuuuaaagagaagacugaaguugaaccauuugaaaauauugaagucuauaaccuaaugugugaucuucuacgcauucaaccagcaccaaacaauggaacccaugguaguuuaaaccaucuucugaaggugccuuuuuaugagccaucccaugcagaggaggugucaaaguuuucuguuuguggcuuugcuaauccauugcccacagagucucuugacuguuucugcccucaccuacaaaauaguacucagcuggaacaagugaaucagaugcuaaaucucacccaagaagaaauaacagcaacagugaaaguaaauuugccauuugggaggccuaggguacugcagaagaacguggaccacugucuccuuuaccacagggaauaugucaguggauuuggaaaagcuaugaggaugcccauguggaguucauacacagucccccaguugggagacacaucgccucugccucccacugucccagacugucugcgggcugaugucaggguuccuccuucugagagccaaaaauguuccuucuauuuagcagacaagaauaucacccacggcuuccucuauccuccugccagcaauagaacaucagauagccaauaugaugcuuuaauuacuagcaauuugguaccuauguaugaagaauucagaaaaaugugggacuacuuccacaguguucuucuuauaaaacaugccacagaaagaaauggaguaaaugugguuaguggaccaauauuugauuauaauuaugauggccauuuugaugcuccagaugaaauuaccaaacauuuagccaacacugauguucccaucccaacacacuacuuuguggugcugaccaguuguaaaaacaagagccacacaccggaaaacugcccuggguggcuggauguccuacccuuuaucaucccucaccgaccuaccaacguggagagcuguccugaagguaaaccagaagcucuuuggguugaagaaagauuuacagcucacauugcccggguccgugauguagaacuucucacugggcuugacuucuaucaggauaaagugcagccugucucugaaauuuugcaacuaaagacauauuuaccaacauuugaaaccacuauuuaacuuaauaaugucuacuuaauauauaauuuacuguauaaaguaauuuuggcaaaauauaagugauuuuuuucuggagaauuguaaaauaaaguuuucuauuuuuccuuaaaaaaaaaa

SEQ ID NO: 3 - RNA, 21 nt, Homo sapiens
aaucacacaggaccucagcag

SEQ ID NO: 4 - RNA, 21 nt, Homo sapiens
cagcugaaucacacaggaccu

SEQ ID NO: 5 - RNA, 21 nt, Homo sapiens
cagcuacguauccaucgugau

SEQ ID NO: 6 - RNA, 21 nt, Homo sapiens
caucgugauggcaucuacaug

SEQ ID NO: 7 - RNA, 21 nt, Homo sapiens
caugguacacauccaggugac

SEQ ID NO: 8 - RNA, 21 nt, Homo sapiens
cagcuuccaccaagguuguac

SEQ ID NO: 9 - RNA, 21 nt, Homo sapiens
caccaagguuguaccauugcc

SEQ ID NO: 10 - RNA, 21 nt, Homo sapiens
caagguuguaccauugccucc

SEQ ID NO: 11 - RNA, 21 nt, Homo sapiens
gagcugcagcugaaucacaca

SEQ ID NO: 12 - RNA, 21 nt, Homo sapiens
gaaucacacaggaccucagca

SEQ ID NO: 13 - RNA, 21 nt, Homo sapiens
gauggcaucuacaugguacac

SEQ ID NO: 14 - RNA, 21 nt, Homo sapiens
uagcugagcugcagcugaauc

SEQ ID NO: 15 - RNA, 21 nt, Homo sapiens
uacguauccaucgugauggca

SEQ ID NO: 16 - RNA, 21 nt, Homo sapiens
uacaugguacacauccaggug

SEQ ID NO: 17 - RNA, 21 nt, Homo sapiens
ucacacaggaccucagcaguu

SEQ ID NO: 18 - RNA, 21 nt, Homo sapiens
gcugaaucacacaggaccuuu

SEQ ID NO: 19 - RNA, 21 nt, Homo sapiens
gcuacguauccaucgugauuu

SEQ ID NO: 20 - RNA, 21 nt, Homo sapiens
ucgugauggcaucuacauguu

SEQ ID NO: 21 - RNA, 21 nt, Homo sapiens
ugguacacauccaggugacuu

SEQ ID NO: 22 - RNA, 21 nt, Homo sapiens
gcuuccaccaagguuguacuu

SEQ ID NO: 23 - RNA, 21 nt, Homo sapiens
ccaagguuguaccauugccuu

SEQ ID NO: 24 - RNA, 21 nt, Homo sapiens
agguuguaccauugccuccuu

SEQ ID NO: 25 - RNA, 21 nt, Homo sapiens
gcugcagcugaaucacacauu

SEQ ID NO: 26 - RNA, 21 nt, Homo sapiens
aucacacaggaccucagcauu

SEQ ID NO: 27 - RNA, 21 nt, Homo sapiens
uggcaucuacaugguacacuu

SEQ ID NO: 28 - RNA, 21 nt, Homo sapiens
gcugagcugcagcugaaucuu

SEQ ID NO: 29 - RNA, 21 nt, Homo sapiens
cguauccaucgugauggcauu

SEQ ID NO: 30 - RNA, 21 nt, Homo sapiens
caugguacacauccagguguu

SEQ ID NO: 31 - RNA, 21 nt, Homo sapiens
uuaguguguccuggagucguc

SEQ ID NO: 32 - RNA, 21 nt, Homo sapiens
uucgacuuaguguguccugga

SEQ ID NO: 33 - RNA, 21 nt, Homo sapiens
uucgaugcauagguagcacua

SEQ ID NO: 34 - RNA, 21 nt, Homo sapiens
uuagcacuaccguagauguac

SEQ ID NO: 35 - RNA, 21 nt, Homo sapiens
uuaccauguguagguccacug

SEQ ID NO: 36 - RNA, 21 nt, Homo sapiens
uucgaaggugguuccaacaug

SEQ ID NO: 37 - RNA, 21 nt, Homo sapiens
uugguuccaacaugguaacgg

SEQ ID NO: 38 - RNA, 21 nt, Homo sapiens
uuuccaacaugguaacggagg

SEQ ID NO: 39 - RNA, 21 nt, Homo sapiens
uucgacgucgacuuagugugu

SEQ ID NO: 40 - RNA, 21 nt, Homo sapiens
uuuaguguguccuggagucgu

SEQ ID NO: 41 - RNA, 21 nt, Homo sapiens
uuaccguagauguaccaugug

SEQ ID NO: 42 - RNA, 21 nt, Homo sapiens
uucgacucgacgucgacuuag

SEQ ID NO: 43 - RNA, 21 nt, Homo sapiens
uugcauagguagcacuaccgu

SEQ ID NO: 44 - RNA, 21 nt, Homo sapiens
uuguaccauguguagguccac

SEQ ID NO: 45 - RNA, 21 nt, Homo sapiens
aaggcagcugcaggaagaagu

SEQ ID NO: 46 - RNA, 21 nt, Homo sapiens
aagaccgaggugauugcugcu

SEQ ID NO: 47 - RNA, 21 nt, Homo sapiens
aauaauccagccugguggcau

SEQ ID NO: 48 - RNA, 21 nt, Homo sapiens
aaccaauguggcugacagcaa

SEQ ID NO: 49 - RNA, 21 nt, Homo sapiens
aagaaccugauuccucuggac

SEQ ID NO: 50 - RNA, 21 nt, Homo sapiens
aaccugauuccucuggacaug

SEQ ID NO: 51 - RNA, 21 nt, Homo sapiens
aaggccugaagcagcggaauu

SEQ ID NO: 52 - RNA, 21 nt, Homo sapiens
aagcgacugcacuaugccaag

SEQ ID NO: 53 - RNA, 21 nt, Homo sapiens
aacaguggcuggcuguuagga

SEQ ID NO: 54 - RNA, 21 nt, Homo sapiens
aauuguggaggaggcaaccau

SEQ ID NO: 55 - RNA, 21 nt, Homo sapiens
aaccaucuucugaaggugccu

SEQ ID NO: 56 - RNA, 21 nt, Homo sapiens
aagaacguggaccacugucuc

SEQ ID NO: 57 - RNA, 21 nt, Homo sapiens
aacguggaccacugucuccuu

SEQ ID NO: 58 - RNA, 21 nt, Homo sapiens
aacaagagccacacaccggaa

SEQ ID NO: 59 - RNA, 21 nt, Homo sapiens
aacguggagagcuguccugaa

SEQ ID NO: 60 - RNA, 21 nt, Homo sapiens
caugucacuuggauuaggccu

SEQ ID NO: 61 - RNA, 21 nt, Homo sapiens
cagcugcaggaagaagugcuu

SEQ ID NO: 62 - RNA, 21 nt, Homo sapiens
caccuguguggaaucaacucg

SEQ ID NO: 63 - RNA, 21 nt, Homo sapiens
cagagucacauggcaucauug

SEQ ID NO: 64 - RNA, 21 nt, Homo sapiens
caaccaauguggcugacagca

SEQ ID NO: 65 - RNA, 21 nt, Homo sapiens
caauguggcugacagcaaugu

SEQ ID NO: 66 - RNA, 21 nt, Homo sapiens
caugccuuacaacggaagugu

SEQ ID NO: 67 - RNA, 21 nt, Homo sapiens
cacuaugccaagaacgucaga

SEQ ID NO: 68 - RNA, 21 nt, Homo sapiens
cagcuggaacaagugaaucag

SEQ ID NO: 69 - RNA, 21 nt, Homo sapiens
cagaagaacguggaccacugu

SEQ ID NO: 70 - RNA, 21 nt, Homo sapiens
gaagacaccuguguggaauca

SEQ ID NO: 71 - RNA, 21 nt, Homo sapiens
gacaccuguguggaaucaacu

SEQ ID NO: 72 - RNA, 21 nt, Homo sapiens
gagagaccagauuagaggcca

SEQ ID NO: 73 - RNA, 21 nt, Homo sapiens
gaccugccaccaguuaucuug

SEQ ID NO: 74 - RNA, 21 nt, Homo sapiens
gagucacauggcaucauugac

SEQ ID NO: 75 - RNA, 21 nt, Homo sapiens
gaagaaccugauuccucugga

SEQ ID NO: 76 - RNA, 21 nt, Homo sapiens
gaaccugauuccucuggacau

SEQ ID NO: 77 - RNA, 21 nt, Homo sapiens
gauuccucuggacaugcaggu

SEQ ID NO: 78 - RNA, 21 nt, Homo sapiens
gaccagucagugccagaguaa

SEQ ID NO: 79 - RNA, 21 nt, Homo sapiens
gauguugauggaaggccugaa

SEQ ID NO: 80 - RNA, 21 nt, Homo sapiens
gaccauggaauggaccagacu

SEQ ID NO: 81 - RNA, 21 nt, Homo sapiens
gacugcacuaugccaagaacg

SEQ ID NO: 82 - RNA, 21 nt, Homo sapiens
gaucaacaguggcuggcuguu

SEQ ID NO: 83 - RNA, 21 nt, Homo sapiens
gaggaggcaaccaugguuaua

SEQ ID NO: 84 - RNA, 21 nt, Homo sapiens
gaagaacguggaccacugucu

SEQ ID NO: 85 - RNA, 21 nt, Homo sapiens
uauccagagucacauggcauc

SEQ ID NO: 86 - RNA, 21 nt, Homo sapiens
uaucauccuucuggcugacca

SEQ ID NO: 87 - RNA, 21 nt, Homo sapiens
uaggagcauggaggcuaucuu

SEQ ID NO: 88 - RNA, 21 nt, Homo sapiens
uacgcauucaaccagcaccaa

SEQ ID NO: 89 - RNA, 21 nt, Homo sapiens
uacugcagaagaacguggacc

SEQ ID NO: 90 - RNA, 21 nt, Homo sapiens
uauccuccugccagcaauaga

SEQ ID NO: 91 - RNA, 21 nt, Homo sapiens
ggcagcugcaggaagaaguuu

SEQ ID NO: 92 - RNA, 21 nt, Homo sapiens
gaccgaggugauugcugcuuu

SEQ ID NO: 93 - RNA, 21 nt, Homo sapiens
uaauccagccugguggcauuu

SEQ ID NO: 94 - RNA, 21 nt, Homo sapiens
ccaauguggcugacagcaauu

SEQ ID NO: 95 - RNA, 21 nt, Homo sapiens
gaaccugauuccucuggacuu

SEQ ID NO: 96 - RNA, 21 nt, Homo sapiens
ccugauuccucuggacauguu

SEQ ID NO: 97 - RNA, 21 nt, Homo sapiens
ggccugaagcagcggaauuuu

SEQ ID NO: 98 - RNA, 21 nt, Homo sapiens
gcgacugcacuaugccaaguu

SEQ ID NO: 99 - RNA, 21 nt, Homo sapiens
caguggcuggcuguuaggauu

SEQ ID NO: 100 - RNA, 21 nt, Homo sapiens
uuguggaggaggcaaccauuu

SEQ ID NO: 101 - RNA, 21 nt, Homo sapiens
ccaucuucugaaggugccuuu

SEQ ID NO: 102 - RNA, 21 nt, Homo sapiens
gaacguggaccacugucucuu

SEQ ID NO: 103 - RNA, 21 nt, Homo sapiens
cguggaccacugucuccuuuu

SEQ ID NO: 104 - RNA, 21 nt, Homo sapiens
caagagccacacaccggaauu

SEQ ID NO: 105 - RNA, 21 nt, Homo sapiens
cguggagagcuguccugaauu

SEQ ID NO: 106 - RNA, 21 nt, Homo sapiens
ugucacuuggauuaggccuuu

SEQ ID NO: 107 - RNA, 21 nt, Homo sapiens
gcugcaggaagaagugcuuuu

SEQ ID NO: 108 - RNA, 21 nt, Homo sapiens
ccuguguggaaucaacucguu

SEQ ID NO: 109 - RNA, 21 nt, Homo sapiens
gagucacauggcaucauuguu

SEQ ID NO: 110 - RNA, 21 nt, Homo sapiens
accaauguggcugacagcauu

SEQ ID NO: 111 - RNA, 21 nt, Homo sapiens
auguggcugacagcaauguuu

SEQ ID NO: 112 - RNA, 21 nt, Homo sapiens
ugccuuacaacggaaguguuu

SEQ ID NO: 113 - RNA, 21 nt, Homo sapiens
cuaugccaagaacgucagauu

SEQ ID NO: 114 - RNA, 21 nt, Homo sapiens
gcuggaacaagugaaucaguu

SEQ ID NO: 115 - RNA, 21 nt, Homo sapiens
gaagaacguggaccacuguuu

SEQ ID NO: 116 - RNA, 21 nt, Homo sapiens
agacaccuguguggaaucauu

SEQ ID NO: 117 - RNA, 21 nt, Homo sapiens
caccuguguggaaucaacuuu

SEQ ID NO: 118 - RNA, 21 nt, Homo sapiens
gagaccagauuagaggccauu

SEQ ID NO: 119 - RNA, 21 nt, Homo sapiens
ccugccaccaguuaucuuguu

SEQ ID NO: 120 - RNA, 21 nt, Homo sapiens
gucacauggcaucauugacuu

SEQ ID NO: 121 - RNA, 21 nt, Homo sapiens
agaaccugauuccucuggauu

SEQ ID NO: 122 - RNA, 21 nt, Homo sapiens
accugauuccucuggacauuu

SEQ ID NO: 123 - RNA, 21 nt, Homo sapiens
uuccucuggacaugcagguuu

SEQ ID NO: 124 - RNA, 21 nt, Homo sapiens
ccagucagugccagaguaauu

SEQ ID NO: 125 - RNA, 21 nt, Homo sapiens
uguugauggaaggccugaauu

SEQ ID NO: 126 - RNA, 21 nt, Homo sapiens
ccauggaauggaccagacuuu

SEQ ID NO: 127 - RNA, 21 nt, Homo sapiens
cugcacuaugccaagaacguu

SEQ ID NO: 128 - RNA, 21 nt, Homo sapiens
ucaacaguggcuggcuguuuu

SEQ ID NO: 129 - RNA, 21 nt, Homo sapiens
ggaggcaaccaugguuauauu

SEQ ID NO: 130 - RNA, 21 nt, Homo sapiens
agaacguggaccacugucuuu

SEQ ID NO: 131 - RNA, 21 nt, Homo sapiens
uccagagucacauggcaucuu

SEQ ID NO: 132 - RNA, 21 nt, Homo sapiens
ucauccuucuggcugaccauu

SEQ ID NO: 133 - RNA, 21 nt, Homo sapiens
ggagcauggaggcuaucuuuu

SEQ ID NO: 134 - RNA, 21 nt, Homo sapiens
cgcauucaaccagcaccaauu

SEQ ID NO: 135 - RNA, 21 nt, Homo sapiens
cugcagaagaacguggaccuu

SEQ ID NO: 136 - RNA, 21 nt, Homo sapiens
uccuccugccagcaauagauu

SEQ ID NO: 137 - RNA, 21 nt, Homo sapiens
uuccgucgacguccuucuuca

SEQ ID NO: 138 - RNA, 21 nt, Homo sapiens
uucuggcuccacuaacgacga

SEQ ID NO: 139 - RNA, 21 nt, Homo sapiens
uuauuaggucggaccaccgua

SEQ ID NO: 140 - RNA, 21 nt, Homo sapiens
uugguuacaccgacugucguu

SEQ ID NO: 141 - RNA, 21 nt, Homo sapiens
uucuuggacuaaggagaccug

SEQ ID NO: 142 - RNA, 21 nt, Homo sapiens
uuggacuaaggagaccuguac

SEQ ID NO: 143 - RNA, 21 nt, Homo sapiens
uuccggacuucgucgccuuaa

SEQ ID NO: 144 - RNA, 21 nt, Homo sapiens
uucgcugacgugauacgguuc

SEQ ID NO: 145 - RNA, 21 nt, Homo sapiens
uugucaccgaccgacaauccu

SEQ ID NO: 146 - RNA, 21 nt, Homo sapiens
uuaacaccuccuccguuggua

SEQ ID NO: 147 - RNA, 21 nt, Homo sapiens
uugguagaagacuuccacgga

SEQ ID NO: 148 - RNA, 21 nt, Homo sapiens
uucuugcaccuggugacagag

SEQ ID NO: 149 - RNA, 21 nt, Homo sapiens
uugcaccuggugacagaggaa

SEQ ID NO: 150 - RNA, 21 nt, Homo sapiens
uuguucucgguguguggccuu

SEQ ID NO: 151 - RNA, 21 nt, Homo sapiens
uugcaccucucgacaggacuu

SEQ ID NO: 152 - RNA, 21 nt, Homo sapiens
uuacagugaaccuaauccgga

SEQ ID NO: 153 - RNA, 21 nt, Homo sapiens
uucgacguccuucuucacgaa

SEQ ID NO: 154 - RNA, 21 nt, Homo sapiens
uuggacacaccuuaguugagc

SEQ ID NO: 155 - RNA, 21 nt, Homo sapiens
uucucaguguaccguaguaac

SEQ ID NO: 156 - RNA, 21 nt, Homo sapiens
uuugguuacaccgacugucgu

SEQ ID NO: 157 - RNA, 21 nt, Homo sapiens
uuuacaccgacugucguuaca

SEQ ID NO: 158 - RNA, 21 nt, Homo sapiens
uuacggaauguugccuucaca

SEQ ID NO: 159 - RNA, 21 nt, Homo sapiens
uugauacgguucuugcagucu

SEQ ID NO: 160 - RNA, 21 nt, Homo sapiens
uucgaccuuguucacuuaguc

SEQ ID NO: 161 - RNA, 21 nt, Homo sapiens
uucuucuugcaccuggugaca

SEQ ID NO: 162 - RNA, 21 nt, Homo sapiens
uuucuguggacacaccuuagu

SEQ ID NO: 163 - RNA, 21 nt, Homo sapiens
uuguggacacaccuuaguuga

SEQ ID NO: 164 - RNA, 21 nt, Homo sapiens
uucucuggucuaaucuccggu

SEQ ID NO: 165 - RNA, 21 nt, Homo sapiens
uuggacgguggucaauagaac

SEQ ID NO: 166 - RNA, 21 nt, Homo sapiens
uucaguguaccguaguaacug

SEQ ID NO: 167 - RNA, 21 nt, Homo sapiens
uuucuuggacuaaggagaccu

SEQ ID NO: 168 - RNA, 21 nt, Homo sapiens
uuuggacuaaggagaccugua

SEQ ID NO: 169 - RNA, 21 nt, Homo sapiens
uuaaggagaccuguacgucca

SEQ ID NO: 170 - RNA, 21 nt, Homo sapiens
uuggucagucacggucucauu

SEQ ID NO: 171 - RNA, 21 nt, Homo sapiens
uuacaacuaccuuccggacuu

SEQ ID NO: 172 - RNA, 21 nt, Homo sapiens
uugguaccuuaccuggucuga

SEQ ID NO: 173 - RNA, 21 nt, Homo sapiens
uugacgugauacgguucuugc

SEQ ID NO: 174 - RNA, 21 nt, Homo sapiens
uuaguugucaccgaccgacaa

SEQ ID NO: 175 - RNA, 21 nt, Homo sapiens
uuccuccguugguaccaauau

SEQ ID NO: 176 - RNA, 21 nt, Homo sapiens
uuucuugcaccuggugacaga

SEQ ID NO: 177 - RNA, 21 nt, Homo sapiens
uuaggucucaguguaccguag

SEQ ID NO: 178 - RNA, 21 nt, Homo sapiens
uuaguaggaagaccgacuggu

SEQ ID NO: 179 - RNA, 21 nt, Homo sapiens
uuccucguaccuccgauagaa

SEQ ID NO: 180 - RNA, 21 nt, Homo sapiens
uugcguaaguuggucgugguu

SEQ ID NO: 181 - RNA, 21 nt, Homo sapiens
uugacgucuucuugcaccugg

SEQ ID NO: 182 - RNA, 21 nt, Homo sapiens
uuaggaggacggucguuaucu

(19) United States
(12) Patent Application Publication
(10) Pub. No.: US 2005/0282168 A1
(43) Pub. Date: Dec. 22, 2005

(54) Cell surface molecules as markers and therapeutic agents against kidney cancers

(75) Inventors: Wei Liu, Sudbury, MA (US); Maryann Whitley, Quincy, MA (US); Donna Slonim, North Andover, MA (US); Steven Howes, Cambridge, MA (US)

Correspondence Address: FITZPATRICK CELLA (WYETH), 30 ROCKEFELLER PLAZA, NEW YORK, NY, 10112-3800, US

(73) Assignee: Wyeth, Madison, NJ (US)

(21) Appl. No.: 10/671,740

(22) Filed: Sep. 29, 2003

(51) Int. Cl. C12Q 1/68; G01N 33/574

(52) U.S. Cl. 435/6; 435/7.23

(57) ABSTRACT

Human cell surface molecules CD70 and CD203c are expressed at higher levels in kidney carcinomas, particularly renal cell carcinomas and clear cell renal cell carcinomas, yet are expressed at low levels in normal kidney and other diseased kidney tissue, and at low levels in other tissues. CD70 and CD203c show specificity towards kidney carcinomas, particularly renal cell carcinomas and clear cell renal cell carcinomas and thus can be used as diagnostic markers and therapeutic targets for these diseases. In addition, antibodies or small molecules against these molecules could be used in treatments towards these diseases.

This invention relates to CD70 and CD203c. In particular, this invention relates to the use of CD70 and CD203c as markers for and therapeutic agents against kidney carcinomas, particularly renal cell carcinoma and clear cell renal cell carcinoma.

### BACKGROUND OF THE INVENTION

1. CD70

CD70 is a cytokine that shows homology with TNF-α, TNF-β as well as the ligand for CD40, and is a type II transmembrane protein. The term “CD70” is interchangeable with the following terms: “CD27 ligand,” “CD27L,” “CD27LG,” “tumor necrosis factor ligand superfamily #7,” “TNFSF7,” and “Ki-24 antigen” (the term “Ki-24” denotes an anti-CD70 monoclonal antibody (MAb)). Each of these terms refers to a genus of polypeptides that is capable of binding CD27 and includes the human form of the polypeptide. Moreover, we understand each of these terms to include (except for Ki-24) both the membrane-bound proteins (which contain a cytoplasmic domain, a transmembrane region, and an extracellular domain), as well as truncated proteins, including soluble CD70, that can still bind CD27. When possible, “CD70” will be utilized herein for consistency.

CD70 is believed to ligate to CD27, thereby initiating the biological signal mediated by CD27, which is constitutively expressed on T cells. In accordance with its role during specific stages of the immune response, normal CD70 expression is very restricted in vivo and is known to be expressed on the surface of activated but not resting B and T lymphocytes. In fact, CD70-CD27 interaction has been found to be important for T cell co-stimulation, natural killer (NK) cell activation and T cell-dependent B cell activation. In addition to its role in controlling T-cell activation, CD27/CD70 ligation contributes to immunity by facilitating effector T cell differentiation.

CD70 expression also has been found in many peripheral T- and B-cell lymphomas, as well as on lymphocytes from chronic B cell lymphocytic leukemia patients. (See Arens, R. et al., “Constitutive CD27/CD70 interaction induces expansion of effector-type T Cells and results in IFN_{γ}-mediated B cell depletion,” Immunity 15: 801-812 (November 2001).) In the majority of these malignancies, CD70 was actually co-expressed with CD27, suggesting that CD27-CD70 interactions could take place in malignant cell populations. CD70 also has been shown to be expressed by thymic carcinoma. (See also Hishima, T. et al., “CD70 expression in thymic carcinoma,” Am. J. Surg. Pathol. 24(5): 742-6 (May, 2000) (Abstract).)

In addition, Bruce Israel et al., disclose that certain cancer cells (e.g., lymphoblastoid cell lines (LCLs), certain Epstein-Barr virus (EBV)-positive and -negative B-cell lymphomas, EBV-positive nasopharyngeal carcinoma) commonly express CD70, and posit that CD70 expression might serve as a marker with which to direct adenovirus vectors to many such cells. (See Israel, B. F. et al., “Enhancement of Adenovirus Vector Entry into CD-70-Positive B-Cell Lines by Using a Bispecific CD70-Adenovirus Fiber Antibody,” J. Virol. 75(11): 5215-5221 (June 2001).)

The cDNA sequence and predicted amino acid sequence of human CD70 are set forth in U.S. Pat. No. 5,573,924, which sequences are incorporated herein by reference in their entirety. These sequences also are found in Goodwin, R. G. et al. “Molecular and Biological Characterization of a Ligand for CD27 Defines a New Family of Cytokines with Homology to Tumor Necrosis Factor,” Cell 73: 447-456 (1993), the sequences of which also are incorporated herein by reference in their entirety. A 240 base pair expressed sequence tag (i.e., EST) for CD70 derived from a renal cell adenocarcinoma tissue sample can be found in the National Center for Biotechnology Information (NCBI) nucleotide database under record number BG420391, the contents of this record number being incorporated herein by reference in its entirety. Other ESTs for CD70 can be found in the NCBI UniGene database under record number UniGene Cluster Hs. 99899.

The nucleotide and predicted amino acid sequences for murine CD70 (“mCD70”) have also been characterized, showing 62% homology at the protein level with its human counterpart, (“hCD70”) in Tesselaar, K. et al., “Characterization of Murine CD70, the Ligand of the TNF Receptor Family Member CD27,” J. Immunol. 159: 4959-4965 (1997), the sequences being incorporated herein by reference in their entirety. (See also Oshima, H. et al., “Characterization of murine CD70 by molecular cloning and MAb,” Int'l Immunol. 10(4): 517-526 (1998), which discloses that there is 57% homology at the amino acid level between mCD70 and hCD70.)

The lymphocyte antigen, CD27, to which CD70 binds, is a cytokine receptor that is found on the surface of most human T lymphocytes and some B lymphocytes (e.g., memory-type B cells). CD27 is a type I transmembrane protein, and is believed to mediate functions that allow survival of activated cells. The cDNA and predicted amino acid sequence of CD27 has been isolated. (See Camerini, D. et al., “The T cell activation antigen CD27 is a member of the nerve growth factor/tumor necrosis factor receptor gene family,” J. Immunol. 147: 3165-3169 (1991)). Both the cDNA and amino acid sequences of CD27 shown in Camerini are incorporated herein by reference. Moreover, murine CD27 (“mCD27”) cDNA and the predicted amino acid sequence of the mCD27 protein are reported in Gravestein, L. A. et al., “Cloning and expression of murine CD27: comparison with 4-1BB, another lymphocyte-specific member of the nerve growth factor receptor family,” Eur. J. Immunol. 23: 943-950 (1993), the sequences being incorporated herein by reference in their entirety. We understand the term CD27 to refer to a genus of polypeptides that is capable of binding of CD70 and includes the human form of the polypeptide.

2. CD203c

CD203c belongs to a series of ectoenzymes that are involved in hydrolysis of extracellular nucleotides, and is interchangeably referred to herein and by those skilled in the art, as: nucleotide pyrophosphatase/phosphodiesterase 3, phosphodiesterase I/nucleotide pyrophosphatase 3, NPP3, E-NPP3, B10 and gp130^{RB13-6}. Previously, CD203c has also been referred to as PDNP3 and Pdnpno, although such terminology is no longer used often. We understand each of these terms to include the human form of the polypeptide, and also to include both the membrane-bound proteins (which contain a cytoplasmic domain, a transmembrane region, and an extracellular domain), as well as truncated proteins, including soluble CD203c, that retain their functionality. When possible, the term “CD203c” will be utilized herein for consistency. The term “97A6” denotes an anti-CD203c monoclonal antibody (MAb). (See Buhring, H. J. et al., “The basophil activation marker defined by antibody 97A6 is identical to the ectonucleotide pyrophosphatase/phosphodiesterase 3,” Blood 97(10): 3303-3305 (May 15, 2001).)

The human nucleotide sequence and predicted amino acid sequence of CD203c are set forth, respectively, in Jin-Hua, P. et al., “Molecular cloning and chromosomal localization of PD-Ibeta (PDNP3), a new member of the human phosphodiesterase I genes,” Genomics 45(2): 412-415 (1997), the sequences being incorporated herein by reference in their entirety. The gene for CD203c has been cloned from humans (i.e., from the colon, prostate, uterus, and basophils), as well as from rat (i.e., from the pancreas, small intestine, liver, embryonic glial precursor cells, and vascular smooth muscle cells).

Nucleotide pyrophosphatases/phosphodiesterases (NPPs) exist as membrane proteins and as soluble proteins in body fluids. NPPs are modular proteins consisting of a short N-terminal intracellular domain, a single transmembrane domain, two somatomedin-Bp-like domains, a catalytic domain, and a C-terminal nuclease-like domain. The catalytic domain of NPPs is conserved from prokaryotes to mammals and is similar to the catalytic domain of other phospho-sulfo-coordinating enzymes such as alkaline phosphatases. Other well characterized NPPs include the mammalian ecto-enzymes NPP1 (PC-1) and NPP2 (autotaxin).

NPP1-3 have been implicated in various processes, such as bone mineralization, signaling by insulin and by nucleotides, and the differentiation and motility of cells. NPP4 and NPP5 have been described as putative nucleotide pyrophosphatases/phosphodiesterases based on their homology with the catalytic domain of NPP1-3. That is, all residues known to be essential to the catalytic activity on NPP1-3 exist in NPP4 and 5; however, actual catalytic activity remains to be verified in these proteins. (See Bollen, M. et al., “Nucleotide phyrophosphatases/phosphodiesterases on the move,” Crit. Rev. Biochem. Mol. Biol. 35(6): 393-432 (2000).)

NPPs release nucleoside 5′-monosphosphates from nucleotides and their derivatives by hydrolyzing pyrophosphate/phosphodiester bonds via a nucleotidylated threonine. They are also known to auto(de)phosphorylate this threonine at the active site via an intrinsic phosphatase activity. The phosphorylated enzyme represents the catalytic intermediate of the phosphatase reaction. NPP3 or CD203c is a type II transmembrane protein that is located only at the apical surface of polarized cells. No obvious endocytosis signals on CD203c have been found to date.

CD203c has been associated with tumorigenesis. For example, rat CD 203c⁺ glial precursor cells have been found to be highly susceptible to ethylnitrosourea (EtNU). (See Blass-Kampmann, S. et al., “gp130RB13-6-positive neural progenitor cells are susceptible to the oncogenic effect of ethylnitrosourea in pre-natal rat brain,” Neuropathol. Appl. Neurobiol. 24(1): 9-20 (February 1998) (Abstract).) Also, mouse fibroblast and rat glioma cells over-expressing CD203c exhibit altered morphologies and invasive properties. (See Deissler, H. et al., “Neural cell surface differentiation antigen gp130^{RB13-6} induces fibroblasts and glioma cells to express astroglial proteins and invasive properties,” FASEB J. 13(6): 657-66 (April 1999).)

3. Renal Cell Carcinoma (RCC)

Renal cell carcinoma (RCC) is the most common malignancy arising in the adult kidney, and is sometimes referred to as renal cell adenocarcinoma. The clinicopathology of the disease is heterogeneous. The disease is subdivided using cytoplasmic features into clear, papillary, granular, spindle, and mixed cell variants, with the clear cell variant being the most common. The clear cell variant of the disease is referred to as clear cell renal cell carcinoma (ccRCC) or clear cell carcinoma, and can also be referred to as clear cell adenocarcinoma. Tumor staging and histological grading is used to grade the severity of the malignancy. Patients that have metastatic RCC (i.e., about 30% of cases) have a life expectancy, on average, of about 12 months. Patients with non-metastatic forms of RCC usually have relapses after surgery and eventually succumb to the disease.

Given that it is beneficial to diagnose a patient's cancer condition as soon as possible so that a suitable therapy can be devised and administered promptly, it is plainly useful to develop a rapid and convenient method for detecting renal cell carcinoma and clear cell renal cell carcinoma in a patient. Furthermore, given the ultimate morbidity of the disease, it would be beneficial to provide new and improved treatment methods and pharmaceutical compositions for patients suffering from or at risk of developing renal cell carcinoma or clear cell renal cell carcinoma.

In order to better understand the variable prognoses of patients diagnosed with clear cell renal cell carcinoma (ccRCC), Takahashi et al. conducted a gene expression profiling using malignant tissue specimens obtained from patients suffering from this disease. Takahashi et al. obtained the gene expression profiles for these specimens and identified common alterations in ccRCC gene expression, as well as expression signatures of ccRCC specific to particular clinical subsets of tumors. (See Takahashi, M. et al., “Gene expression profiling of clear cell renal cell carcinoma: Gene identification and prognostic classification,” Proc. Natl. Acad. Sci. 98(17): 9754-9759 (Aug. 14, 2001).)

In supplemental material, Takahashi et al. disclose their results from microarray experiments evaluating 32 commonly up-regulated and commonly down-regulated genes in ccRCC, including phosphodiesterase I/nucleotide pyrophosphatase 3 (i.e., having GenBank accession no. AA678335). That is, the gene encoding CD203c (i.e., phosphodiesterase I/nucleotide pyrophosphatase 3) was noted as being up-regulated in 84.0% of the 29 ccRCC tissue specimens that were studied. (See Supplementary material for Takahashi et al. (Aug. 7, 2001) Proc. Natl. Acad. Sci. USA, 10.1073/pnas.171209998.) However, Takahashi et al. did not ascribe any importance to the finding that CD203c, in particular, was up-regulated. Moreover, of the commonly up-regulated genes identified by Takahashi et al. in ccRCC, CD203c was up-regulated in the smallest percentage of samples. That is, the other genes were up-regulated in 85.7% to 100% of the samples studied.

### SUMMARY OF THE INVENTION

Surprisingly, we have found that the cell surface molecules CD70 and CD203c can be used as markers for and as therapeutic agents towards kidney carcinomas, particularly renal cell carcinoma and clear cell renal cell carcinoma. This is because such cell surface molecules are found in unusually high amounts in renal cell carcinoma and clear cell renal cell carcinoma tissue, as compared to other tissue types.

Accordingly, it is an object of this invention to use CD70 and CD203c individually, and in combination, as markers for renal cell carcinoma and clear cell renal cell carcinoma.

It is a further object of this invention to cause relatively specific killing of renal cell carcinoma and clear cell renal cell carcinoma tissues using approaches that target CD70 or CD203c. This is possible since CD70 is normally only expressed on activated B and T lymphocytes, and is not detectable to a great extent in other tissue types. Furthermore, CD70 and CD203c are not normally detectable in elevated amounts in kidney tissue.

Accordingly, in one aspect, the present invention provides a method of diagnosing kidney cancer in a mammalian patient comprising the steps of: taking a sample of body fluid or tissue from the patient; detecting the amount of CD70 that is present in the patient sample; and comparing the amount of CD70 in the patient sample as against the amount of CD70 in a suitable, normal mammalian body fluid or tissue sample acting as a control, wherein an elevated amount of CD70 in the patient sample compared to the control suggests that the patient has kidney cancer.

In yet another aspect, the invention provides a method of diagnosing kidney cancer in a mammalian patient comprising the steps of: taking a sample of body fluid or tissue from the patient; detecting the amount of CD203c that is present in the patient sample; and comparing the amount of CD203c in the patient sample as against the amount of CD203c in a suitable, normal mammalian body fluid or tissue sample acting as a control, wherein an elevated amount of CD203c in the patient sample compared to the control suggests that the patient has kidney cancer.

In still another aspect, the invention provides an assay to detect the presence of renal cell carcinoma cells or clear cell renal cell carcinoma cells in a human patient comprising the steps of: taking a kidney tissue sample or a blood sample from the patient; detecting the amounts of CD70 and CD203c that are present in the patient sample; and comparing the amounts of CD70 and CD203c in the patient sample as against the amounts of CD70 and CD203c found in a suitable, normal kidney tissue sample or blood sample acting as a control, wherein amounts of CD70 and/or CD203c in the patient sample that are higher than normal, as compared to the control, suggest that the patient is suffering from renal cell carcinoma or clear cell renal cell carcinoma.

In another aspect, the invention provides a pharmaceutical composition comprising: a hybrid molecular structure, itself comprising a molecule that specifically targets CD70 linked to a cellular killing agent; and a pharmaceutically acceptable carrier, wherein the composition destroys malignant kidney tissue.

In yet another aspect, the invention provides a pharmaceutical composition comprising: a hybrid molecular structure itself comprising a molecule that specifically targets CD203c linked to a cellular killing agent; and a pharmaceutically acceptable carrier, wherein the composition destroys malignant kidney tissue.

In still another aspect, the invention provides a method of treating a human patient that has or is at risk of developing renal cell carcinoma or clear cell renal cell carcinoma using a targeted drug delivery approach comprising: preparing an immunoconjugate comprising a cellular killing agent linked to a monoclonal antibody directed against CD70 or CD203c; and administering the immunoconjugate to the patient in a pharmaceutically effective dose.

In another aspect, the invention provides a method of inhibiting the growth of a renal cell carcinoma tumor or a clear cell renal cell carcinoma tumor comprising: preparing a hybrid molecular structure, itself comprising a molecule that specifically targets CD70 and/or CD203c linked to a cellular killing agent; and delivering to the tumor a pharmaceutically effective amount of the hybrid molecular structure.

In yet another aspect, the invention provides a method of treating a human patient that has or is at risk of developing renal cell carcinoma or clear cell renal cell carcinoma comprising: administering directly or indirectly to the patient's kidneys a pharmaceutically effective dose of a preparation, such as: an antibody to CD70 and/or CD203c that is capable of inducing cell death; an antibody to CD70 and/or CD203c that is linked to a cellular killing agent; a peptide fragment that exhibits affinity for CD70 and/or CD203c and that is capable of inducing cell death; or a synthetic composition that exhibits affinity for CD70 and/or CD203c and that is capable of inducing cell death.

Finally, in still another aspect, the invention provides a method of reducing or stopping the growth of malignant kidney tissue in a mammalian patient comprising: reducing the levels of CD70 and/or CD 203c in the patient.

### BRIEF DESCRIPTION OF THE DRAWINGS

FIG. 1A illustrates the percentage of tissue samples in which CD70 transcripts were found in various enumerated kidney tissues;

FIG. 1B illustrates the distribution of hybridization intensities for CD70 transcripts in the same kidney tissue samples considered in FIG. 1A;

FIG. 2A illustrates the percentage of tissue samples in which CD70 transcripts were found in various normal and cancerous tissues;

FIG. 2B illustrates the distribution of hybridization intensities for CD70 transcripts in the same tissue samples considered in FIG. 2A;

FIG. 3 shows a chart depicting the distribution of CD70 transcripts expressed in normal human tissues;

FIG. 4 shows a chart depicting the distribution of CD70 transcripts expressed in malignant human tissues;

FIG. 5A illustrates the percentage of tissue samples in which CD203c transcripts were found in various enumerated kidney tissues;

FIG. 5B illustrates the distribution of hybridization intensities for CD203c transcripts in the same kidney tissue samples considered in FIG. 5A;

FIG. 6A illustrates the percentage of tissue samples in which CD203c transcripts were found in various normal and cancerous tissues;

FIG. 6B illustrates the distribution of hybridization intensities for CD203c transcripts in the same tissue samples considered in FIG. 6A;

FIG. 7 shows a chart depicting the distribution of CD203c transcripts expressed in normal human tissues; and

FIG. 8 shows a chart depicting the distribution of CD203c transcripts expressed in malignant human tissues.

### DETAILED DESCRIPTION OF THE INVENTION

As noted earlier, our invention relates to human cell surface molecules CD70 and CD203c as markers for and therapeutic agents towards kidney carcinomas, particularly renal cell carcinomas and clear cell renal cell carcinomas. These molecules are expressed at higher levels in renal cell carcinomas and clear cell renal cell carcinomas, yet are expressed at low levels in normal kidney tissue and other diseased kidney tissue (including tissues exhibiting conditions such as chronic inflammation, cyst, glomerulosclerosis, oncocytoma, transitional cell carcinoma, and Wilms tumors). Furthermore, these molecules are expressed at low levels in other tissues examined (e.g., breast, colon, lung, liver, pancreas, prostate, and stomach tissues). CD70 and CD203c show specificity towards renal cell carcinomas and clear cell renal cell carcinomas and thus can be used as diagnostic markers for these diseases. In addition, antibodies or small molecules that target these molecules could be used in treatments towards these diseases.

Our surprising invention arose from “data mining”-type research that we undertook using a combination of research tools, including oligonucleotide microarrays available from Affymetrix Inc. (Santa Clara, Calif.) and databases of biological information available from Gene Logic Inc. (Gaithersburg, Md.). These technologies and their application to our research are described further below.

Affymetrix oligonucleotide microarrays (commercially labeled GeneChips®) are widely used to measure the abundance of mRNA molecules in biological samples. Microarrays (or arrays) provide a method for the simultaneous monitoring of the expression levels of many genes in parallel. Because the oligonucleotide probes for each gene are selected and synthesized at specific locations on the array, the hybridization patterns and intensities provide direct indications of the gene identity and relative amount without the need for additional experimentation.

Key to the array design of microarrays is the perfect match/mismatch probe strategy. (The oligonucleotides attached to the chip are referred to as “probes,” because they serve to “probe” or “interrogate” the sample complimentary RNA (cRNA) that is used for testing.) For each probe designed to be perfectly complementary to a target sequence, a partner probe is generated that is identical except for a single base mismatch in its center. Thus, probe pairs are created, called the perfect match probe (PM) and the mismatch probe (MM). (A probe set is a set of probes that are designed to detect one transcript, usually 16-20 probe pairs.) The MM serves as a control for the hybridization specificity of the PM. The PM and MM allow for the quantitation and subtraction of signals caused by non-specific cross-hybridization. The difference in hybridization signals between PM and MM is the parameters used to indicate specific target abundance.

Matrices and algorithms are then used to generate meaningful information from the intensity data obtained from the microarray hybridizations. In an absolute analysis, an experimenter can determine whether a transcript is “present” (such that there is a “present call”) or “absent” (such that there is an “absent call”) by counting the number of probe pairs where the intensity of the PM exceeds the intensity of the MM. An experimenter can also obtain the average difference value which is a quantitative measure of the absolute abundance for each measured transcript in sample. The average difference is directly related to the level of expression of the transcript. More specifically, the average difference is an average of the differences between every PM probe cell and its control MM probe cell in a probe set.

More information on how to analyze data from microarrays and about the data parameters described briefly in the preceding paragraphs can be obtained, for example, from the GeneChip® 3.1 Expression Analysis Algorithm Tutorial Manual found in the Affymetrix GeneChip® Expression Analysis Manual, the entire content of which is herein incorporated by reference.

The data mining power of microarrays is encompassed in another research tool, namely BioExpress™, a comprehensive database of biological information from Gene Logic Inc. (Gaithersburg, Md.), which we used for the data mining that led to our invention described herein. The BioExpress™ database contains broad and in-depth information regarding gene expression in a wide range of normal and diseased human tissues, tissues from experimental animals and human and animal cell lines. Specifically, we used the portion of the BioExpress™ database that contains information generated by using GeneChip® Human Genome U95 Arrays to identify gene expression in a broad range of normal and diseased human tissue samples.

The GeneChip® Human Genome U95 Set contains five arrays that represent more than 60,000 full-length genes and EST clusters. The first array in the set is the HG U95Av2 Array and contains about 12,000 full-length gene sequences that were previously characterized in terms of function or disease association. Arrays B, C, D and E (HG-U95B, HG-U95C, HG-U95D and HG-U95E) contain probes interrogating 50,000 clusters and are comprised of EST sequences. The probe pair in the GeneChip® Human Genome U95 Set that measures transcript for CD70 (i.e., the “qualifier”) is denoted as “34054_at.” Similarly, the “qualifier” for CD203c is “89860_at.”

Consequently, the data output from such a database is akin to an “electronic Northern blot” in that information is obtained about gene expression, but at a very rapid pace since gene expression in numerous samples can be tested simultaneously. GX2000 is the user interface that is used to access the data from the BioExpress™ database.

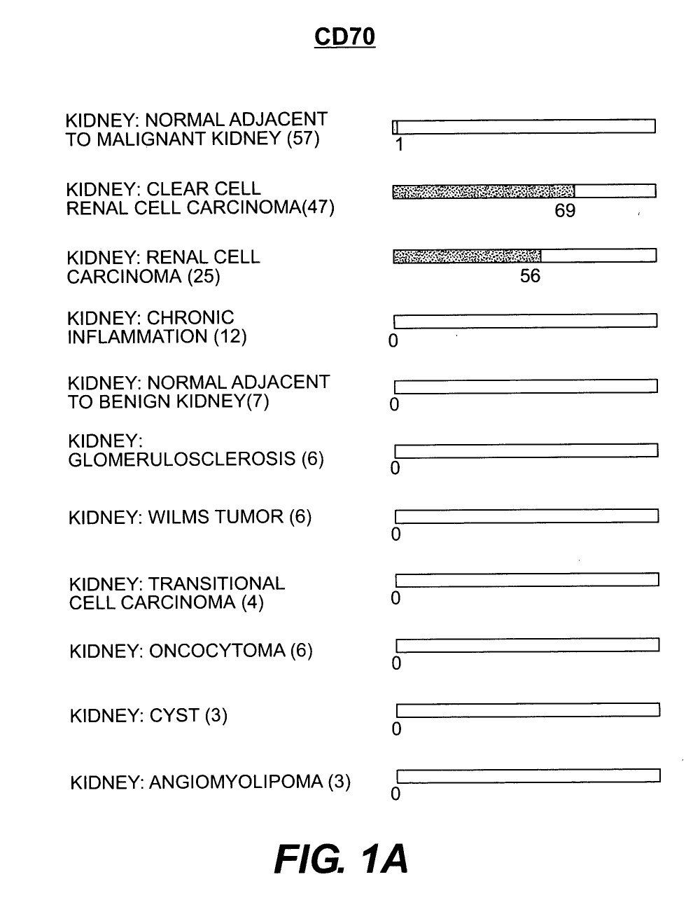

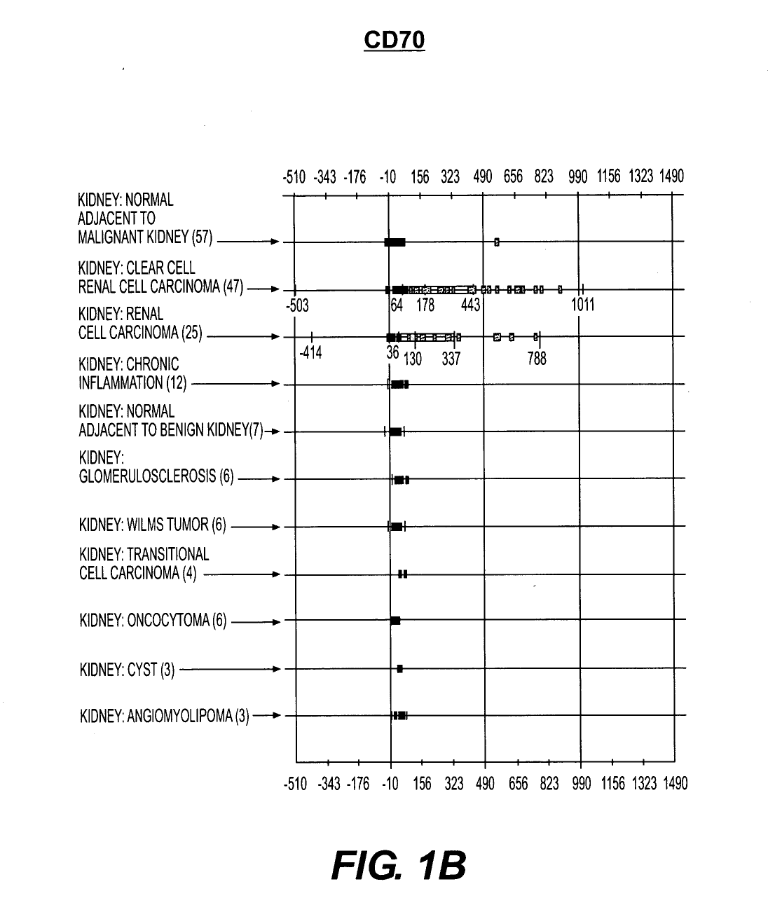

FIGS. 1A to 8 illustrate that CD70 and CD203c are surprisingly found in elevated amounts in renal cell carcinoma and clear cell renal cell carcinoma, as opposed to in other malignant or normal tissues. This is unexpected since we would expect that these cell-surface molecules would be present mostly in the lymphatic system and in lower amounts.

FIGS. 1A to 4 provide surprising information regarding the presence of CD70 in renal cell carcinoma and clear cell renal cell carcinoma tissues as opposed to other types of human tissues. The data set forth in each of these figures are described in more detail below.

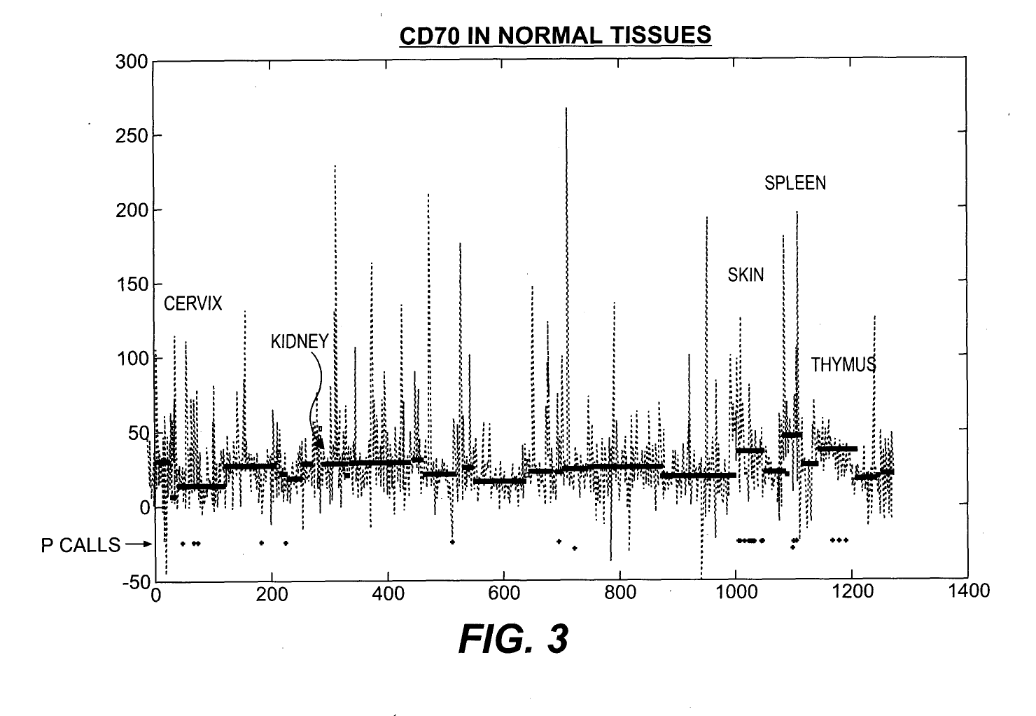

In FIGS. 1B, 2B, 5B, and 6B, the x-axis on the plots denote average difference values that have been normalized whereby the time means of the AD for the entire array equals 100 to allow intensity expression values from different microarray experiments to be compared. FIGS. 3, 4, 7 and 8 also depict “average difference” units but on the y-axis. All average difference values referred to specifically either here or in the figures have been rounded down to the nearest whole number.

More specifically, FIG. 1A shows the percentage of tissue samples in which CD70 transcripts were found, i.e., called “present”, in various enumerated kidney tissues. Directly to the right of each tissue type in parentheses is the number of samples assayed. Accordingly, FIG. 1A illustrates that substantially only renal cell carcinoma and clear cell renal cell carcinoma tissues showed present calls, thereby evidencing expression of CD70. Absent calls were reported for CD70 for other tissue samples (with the exception of one normal tissue sample found adjacent to malignant tissue which exhibited a present call). Specifically, 69% of 47 clear cell renal cell carcinoma samples tested expressed CD70, whereas, 56% of 25 renal cell carcinoma samples tested expressed CD70.

FIG. 1B shows a box plot, whereby the median and standard deviation values were calculated in log-tranformed space and displayed on a linear axis, of the distribution of hybridization intensities (i.e., average difference (AD) values) for CD70 transcripts in the same kidney tissue samples considered in FIG. 1A. As noted earlier, the AD value is directly related to the level of expression of the transcript. Because the AD is an average of the differences in intensity of gene expression between every PM probe cell and its control MM probe cell in a probe set, an AD value is always obtained and there is a distribution of AD values for each tissue type.

Consequently, FIG. 1B shows a distribution of AD values for CD70 expression in the various tissue samples examined. While the spread of the AD values for CD70 expression in most kidney tissue types is very tight around a small AD value which is close to 0, the spread of AD values for RCC and ccRCC is comparatively very broad. For the 47 clear cell renal cell carcinoma tissue samples considered, the median AD value was 178, 64 and 443 were 1 standard deviation away from this midpoint in values, and −503 and 1011 were AD values that were 3 standard deviations away. For the 25 renal cell carcinoma tissue samples considered, the median AD value was 130, 36 and 337 were 1 standard deviation away from this midpoint in values, and −414 and 788 were AD values that were 3 standard deviations away. Thus, even considering the median AD value alone, CD70 appears to be present in greater amounts in clear cell renal cell carcinoma and renal cell carcinoma tissues, than in the other kidney tissue samples considered.

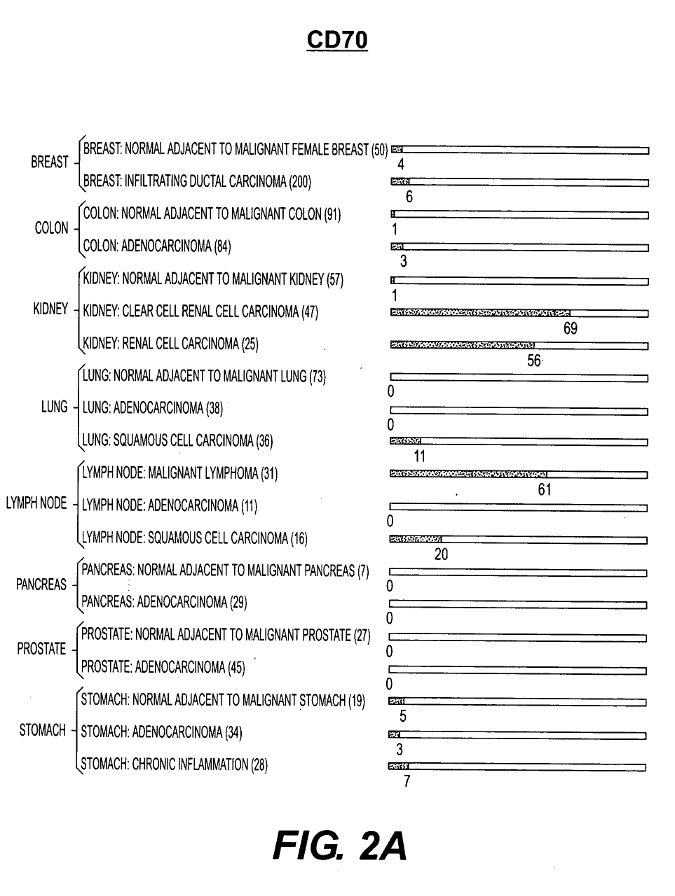

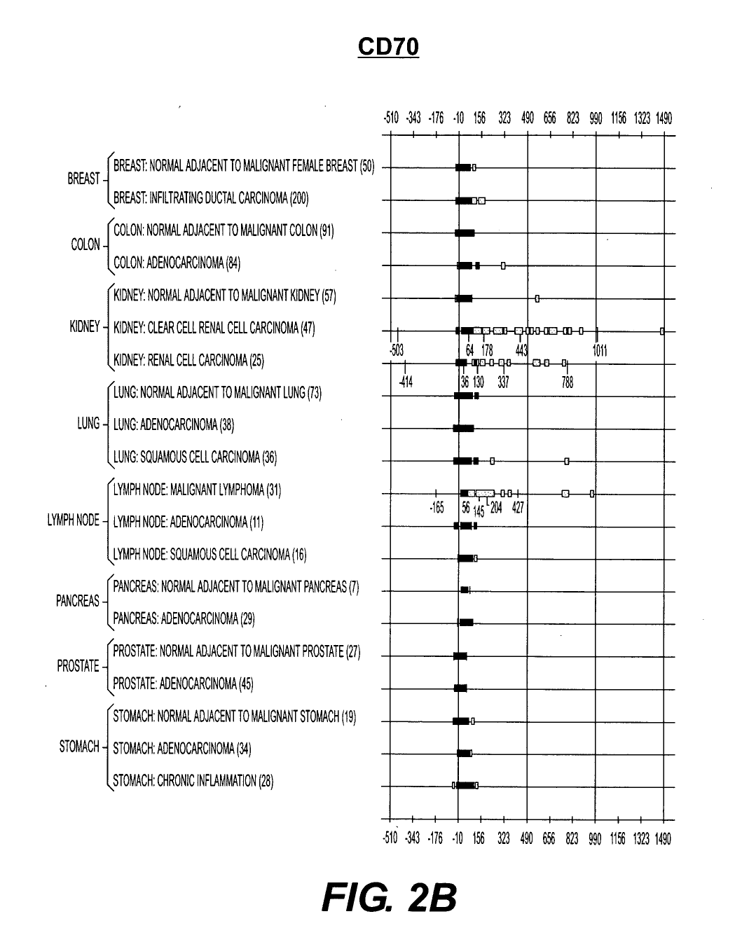

Similar data is provided in FIGS. 2A and 2B for various types of human tissue studied. Namely, FIG. 2A shows the percentage of tissue samples in which CD70 transcripts were found in various tissue samples in the body, mostly cancerous. For most tissue types, the percentage of samples exhibiting CD70 transcripts (i.e., with present call values) was small or zero. However, in clear cell renal cell carcinoma and renal cell carcinoma tissue types, the percentage of samples exhibiting present call values for CD70 was, respectively, 69% and 56%. Given that CD70 is a cell-surface molecule that is a participant in immune system reactions, it is not surprising that the malignant lymphoma tissues showed present call values for CD70 expression in a high percentage of samples.

As with FIG. 1B, FIG. 2B shows a box plot, whereby the median and standard deviation values were calculated in log-transformed space and displayed on a linear axis, of the hybridization intensities (i.e., average difference (AD) values) for CD70 transcripts in the same tissue samples considered in FIG. 2A. As seen in FIG. 1B, for the 47 clear cell renal cell carcinoma tissue samples considered, the median AD value was 178, and for the 25 renal cell carcinoma tissue samples considered, the median AD value was 130. Similar types of outlier values as found in FIG. 1B are also included in FIG. 2B. Consequently, again as in FIG. 1B, considering both the spread of AD value data points and the median AD values, CD70 is present in greater amounts in clear cell renal cell carcinoma and renal cell carcinoma, than in most of the other human tissue samples considered. While malignant lymphoma tissue not surprisingly also had elevated AD values as compared to other malignant tissue in the figure, the spread of those values was not as broad as the spread in the ccRCC and RCC samples considered.

FIG. 3 shows a chart depicting the distribution of CD70 transcripts expressed in normal human tissues. The x-axis represents various human tissue samples listed in alphabetical order (i.e., about 1300 samples considered in total); whereas the y-axis denotes hybridization intensities (i.e., average difference (AD) values) for CD70 expression. Although the spikes showing large AD values may be of interest, these may also be outliers that are not significant. Thus, other parameters shown in FIG. 3 must also be considered.

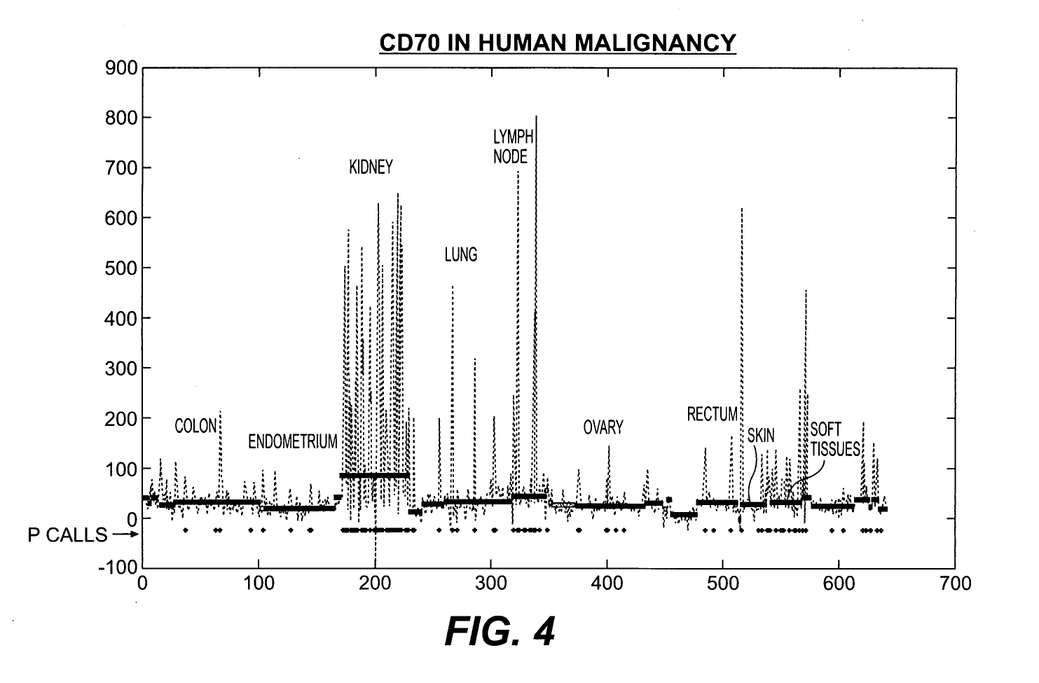

In particular, the horizontal bars group together samples from one type of tissue. The level of that bar is a measure of the median AD value for the tissue type. The higher the level of the bar, the greater the median level of expression of CD70 in the tissue type. Considering FIG. 3 in this light, at the median, normal cervical tissue shows a low amount of CD70 expression, and normal skin, spleen and thymus show higher levels of CD70 expression. However, it should be noted that, on the y-axis, the AD values only go up to 300 which is substantially lower than the AD values in FIG. 4 which go up to 900. Further, in FIG. 3, normal kidney samples are samples 290 to 332 on the x-axis and are also collectively identified by the term “kidney”. The median AD value for these samples is about 27, and is lower than the median for, for example, normal skin, spleen and thymus tissue.

Finally, also presented in FIG. 3 are the present calls (i.e., “P calls”) for CD70 in the various tissue samples, which denote presence or absence of CD70 transcripts. The more dots there are under a particular tissue type, the higher the number of present calls (or “P-calls”) for CD70 expression in the particular tissue. Consequently, the normal skin tissue samples seemed to show the highest amount of P-calls. The “P-calls” should be considered particularly carefully in those instances where the graph shows soaring AD levels in a particular type of tissue. In those instances where the AD value in a tissue type is high but the number of P-calls is small, the high AD value is more likely an indication of an outlier rather than a significant finding as to CD70 expression in a tissue type.

FIG. 4 shows a chart depicting the distribution of CD70 transcripts expressed in malignant human tissues. The x-axis shows the malignant tissue samples considered in alphabetical order (i.e., about 650 in total), and the y-axis depicts the AD values for each tissue sample (i.e., with AD values ranging no higher than about 800). The collection of malignant kidney tissue samples is at samples 171 to 229 and is identified by the term “kidney.” This group includes 59 malignant kidney tissue samples, most of which (i.e., 55 out of 59) are either renal cell carcinoma or clear cell renal cell carcinoma tissues. Also, as in FIG. 3, the bars across the graphs depict tissue samples of one type and the bars' levels show the median AD value for CD70 expression in the samples. Furthermore, the dots below the graph denote present call values for the tissue samples.

Surprisingly, FIG. 4 illustrates that the AD value for malignant kidney tissue is about 82 at the median, and is higher than the median AD value for any of the other tissues. This median AD value is also higher than the median AD values for CD70 expression in all of the normal tissue samples considered in FIG. 3 (which appear to be no higher than 50 in any instance). Furthermore, the concentration of P-calls is greatest among malignant kidney tissue samples. Not surprisingly, malignant lymph node tissue (being a tissue of the immune system) shows, at the median, slightly higher AD values than other tissues. But, the median AD values for malignant lymph node tissue are not as high as the median AD values for malignant kidney tissue. Since, as noted above, most of the malignant kidney tissues are actually RCC or ccRCC tissues, the data from FIG. 4 reveals, in a striking manner, how the elevated levels of CD70 expression can serve as a marker for the presence of such diseased tissue.

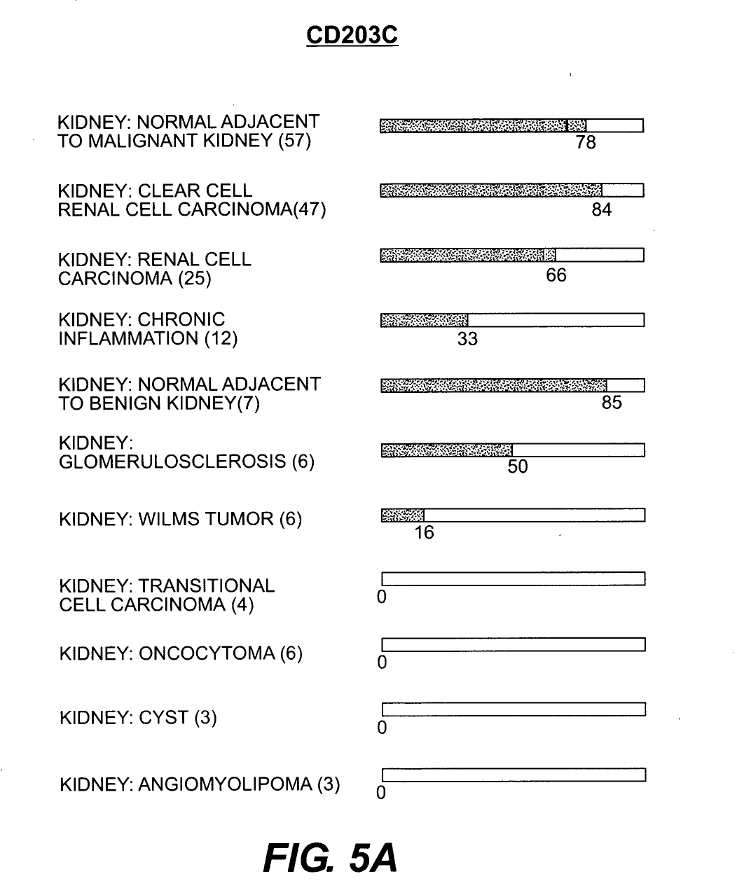

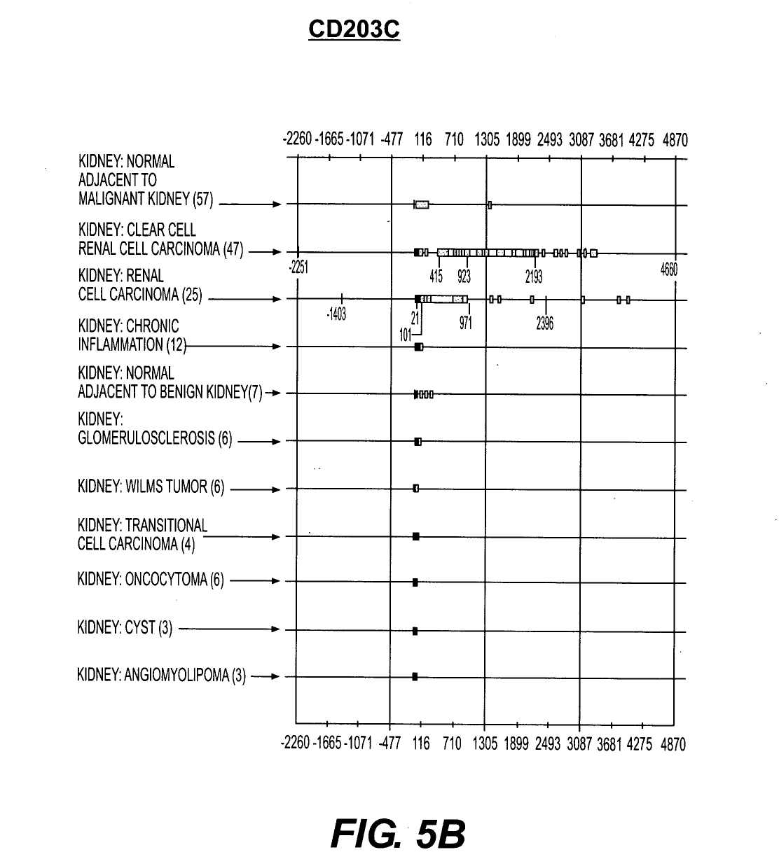

FIGS. 5A to 8 provide surprising information regarding the presence of CD203c in renal cell carcinoma and clear cell renal cell carcinoma tissues as opposed to other types of human tissues. Since the type of information presented in these figures regarding CD70 parallels the data presented in FIGS. 1A to 4 for CD70, we will not describe the data in such great detail here. However, the most salient aspects of the data for CD203c expression in human tissue are considered below.

FIG. 5A shows the percentage of tissue samples in which CD203c transcripts were found in various enumerated kidney tissues. Specifically, FIG. 5A suggests that there are a number of kidney tissue samples expressing CD203c (i.e., exhibiting present call values). That is, not only do substantial percentages of clear cell renal cell carcinoma and renal cell carcinoma tissue samples reveal present calls for CD203c expression (i.e., at 84% and 66%, respectively), but also, 78% of normal kidney tissue samples found adjacent to malignant kidney tissue, 85% of normal kidney tissue samples found adjacent to benign kidney tissue, and 50% of glomerulosclerosis tissue samples reveal present calls for CD203c expression. Consequently, present calls alone do not reveal distinguishing CD203c expression information as regards clear cell renal cell carcinoma and, more broadly, renal cell carcinoma.

FIG. 5B shows a box plot, whereby the median and standard deviation values were calculated in log-transformed space and displayed on a linear axis, of the hybridization intensities (i.e., average difference (AD) values) for CD203c transcripts in the same kidney tissue samples considered in FIG. 5A. The data presentation in this figure reveals that, in many of the samples considered in FIG. 5A yielding present calls for CD203c expression, most tissue samples, in fact, expressed extremely low levels of CD203c. This is apparent from FIG. 5B since the average difference values denoting CD203c expression for the samples of most of the tissue types were tightly crowded around a fairly low average difference value. However, surprisingly, the range of CD203c expression in clear cell renal cell carcinoma and renal cell carcinoma was substantially broader than for other kidney tissue samples. Thus, the overall pattern of CD203c expression shows an increase amongst ccRCC and RCC samples. Similar types of median and outlier values as found in FIGS. 1B and 2B are also included in FIG. 5B. For example, the median AD value for ccRCC is 923, whereas this value for RCC is 101.

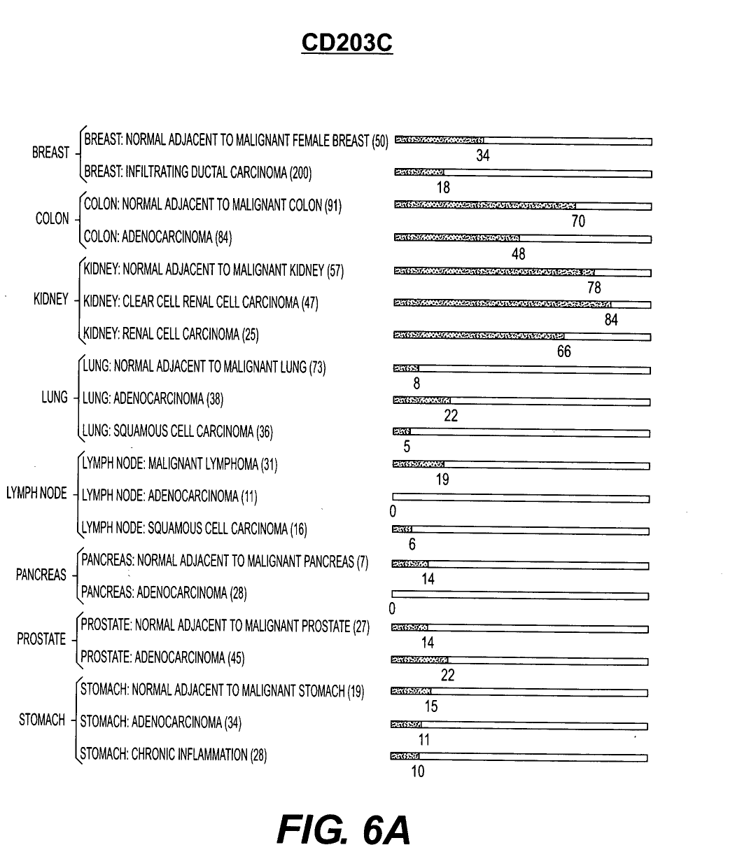

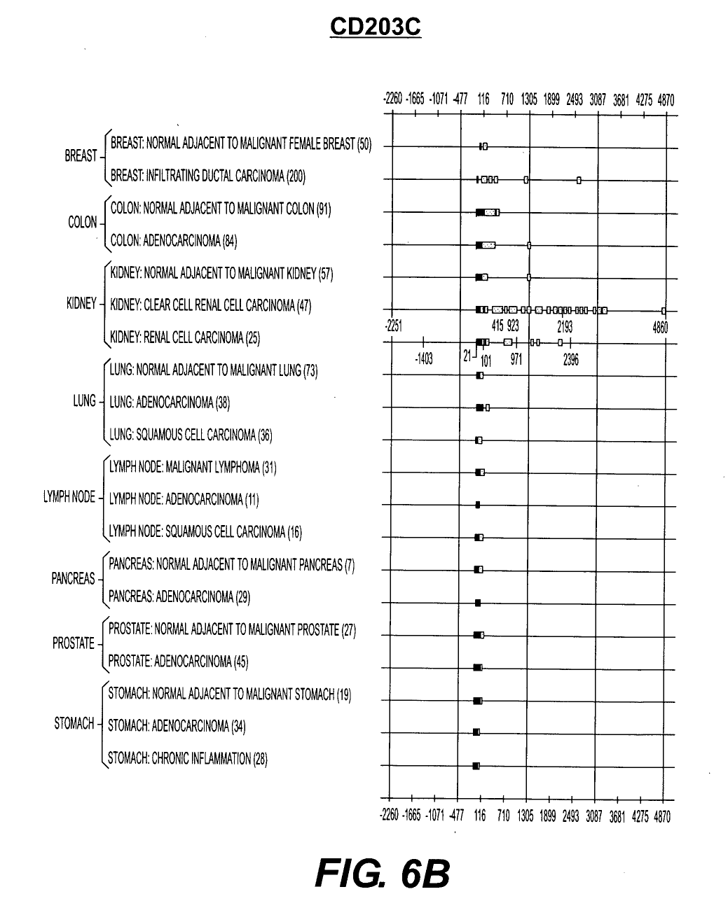

FIG. 6A shows the percentage of tissue samples in which CD203c transcripts were found amongst various types of tissue samples, mostly cancerous. As in FIG. 5A, there appear to be a number of tissue samples expressing CD203c (i.e., exhibiting present call values for CD203c transcripts.) That is, not only do substantial percentages of clear cell renal cell carcinoma and renal cell carcinoma tissue samples reveal present calls for CD203c expression (i.e., at 84% and 66%), but, for example, 70% of normal colon tissue samples found adjacent to malignant colon tissue and 78% of normal kidney tissue samples found adjacent to malignant kidney tissue also reveal present calls for CD203c expression. Consequently, again present calls alone do not reveal distinguishing CD203c expression information as regards clear cell renal cell carcinoma and renal cell carcinoma vis-a-vis other non-kidney-type tissue samples.

FIG. 6B shows a box plot, whereby the median and standard deviation values were calculated in log-transformed space and displayed on a linear axis, of the hybridization intensities (i.e., average difference values) for CD203c transcripts in the same cancerous tissue samples considered in FIG. 6A. Like FIGS. 5A and 5B, the data presentation in FIG. 6B reveals that, in many of the samples considered in FIG. 6A yielding present calls for CD203c expression, most tissue samples, in fact, expressed extremely low levels of CD203c. CD203c expression is substantially higher in clear cell renal cell carcinoma and renal cell carcinoma than in the other selected human tissues. As in FIG. 5B, the median AD value for clear cell renal cell carcinoma is 923, whereas, the median AD value for renal cell carcinoma is 101. Furthermore, instead of the collection of AD values being crowded around one low AD value, the AD values for CD203c expression in ccRCC and RCC are spread out and show a tendency for high levels of expression.

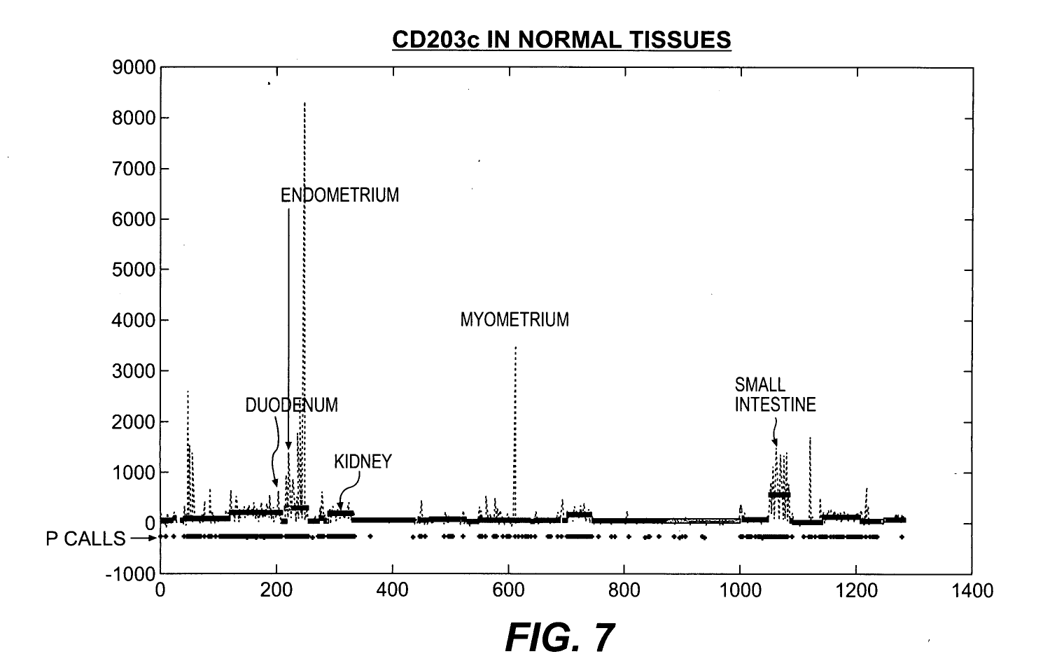

FIG. 7 shows a chart depicting the distribution of CD203c transcripts expressed in normal human tissues. This chart parallels FIG. 3 for CD70 expression in normal tissues; however, of note are the higher AD values that go to 9000 in this chart for CD203c (as opposed to 300 as was the case for CD70). As would be expected based on the data in FIGS. 5A and 6A, present calls or “P calls” are numerous in various normal tissue samples. However, based on the bars in FIG. 7, only in normal small intestine tissue do we see markedly higher median AD values (i.e., about 540) than in other tissues. As in FIG. 3, the normal kidney samples are samples 290-332 on the x-axis and are also collectively identified by the term “kidney.” The median AD value for these samples is about 161.

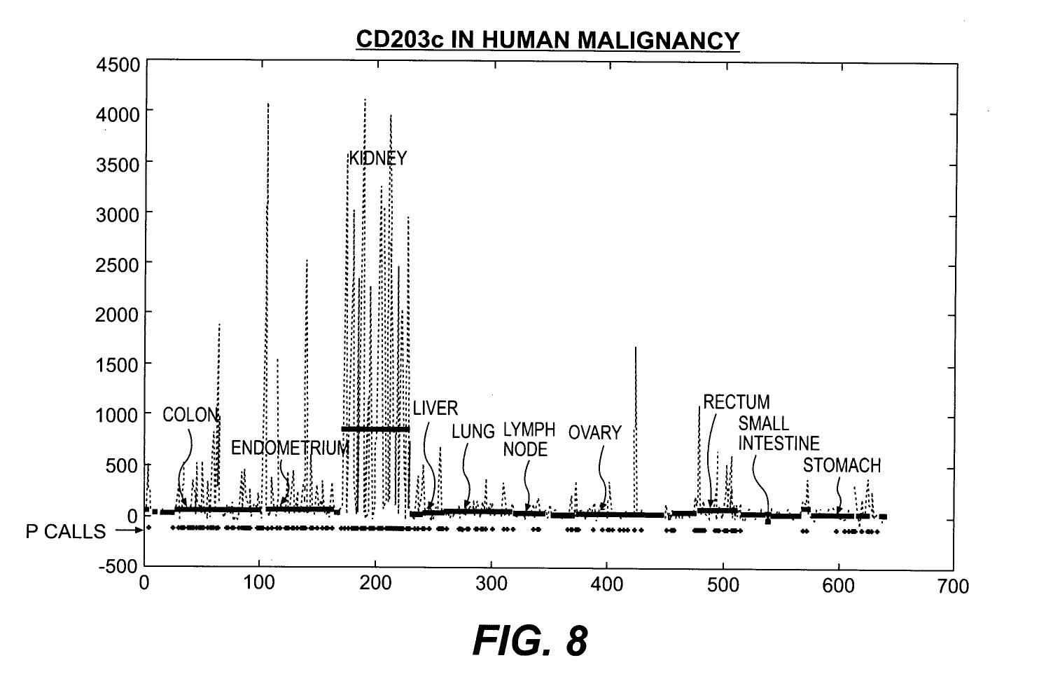

Finally, FIG. 8 shows a chart depicting the distribution of CD203c transcripts expressed in malignant human tissues. AD values on the y-axis of this chart reach 4500. Whereas the median AD values for most of the human malignant tissues are low (i.e., near 0) and yet the present call or “P-call” values are high, the median AD value for kidney tissues is 852-well above the values for any of the other malignant tissue samples. Even the median AD value for small intestine tissue which was high in normal tissue is near 0 among malignant small intestine tissue samples (i.e., at samples 538-541). Furthermore, of the malignant kidney tissue samples at samples 171 to 229, most of these samples (i.e., 55 out or 59) are either ccRCC and RCC tissue samples. Consequently, FIG. 8 supports our finding that CD203c can be used as a marker for kidney cancers, particularly renal cell carcinoma and clear cell renal cell carcinoma, because CD203c expression is up-regulated in such tissues as compared to normal kidney tissue, and is more abundant in such tissues as compared to the other malignant tissues examined.

The up-regulation of CD70 and CD203c expression in ccRCC and, more broadly, RCC as compared to in normal kidney tissue is also apparent from the data set forth in Table 1 below. This data provides AD values for normal and diseased kidney tissue obtained from a subsequent analysis of data available from the BioExpress™ database. As can be seen from the table, the median value for CD70 expression in ccRCC and RCC is, respectively, about 5.4 times (i.e., 178/33) and about 3.9 times (i.e., 130/33) greater than the median amount of CD70 expression in normal kidney tissue samples found adjacent to malignant kidney tissue. Similarly, the median value for CD203c expression in ccRCC and RCC is, respectively, about 15.8 times (i.e., 886/56) and about 1.8 times (i.e., 101/56) greater than the median amount of CD203c expression in normal kidney tissue found adjacent to malignant kidney tissue. Moreover, the median AD values for both CD70 and CD203c expression are higher in ccRCC and RCC tissue types than for any other tissue type.

TABLE 1

| Kidney | Percentile | CD70 | CD203c |
|---|---|---|---|
| Tissue Type | Value Type | Expression | Expression |
| Normal kidney | lower 25% | 20 | 25 |
| tissue adjacent to | median | 33 | 56 |
| malignant kidney | upper 75% | 47 | 78 |
| tissue (51 samples) | | | |
| Kidney clear cell | lower 25% | 64 | 356 |
| renal cell | median | 178 | 886 |
| carcinoma tissue | upper 75% | 447 | 2259 |
| (45 samples) | | | |
| Kidney renal cell | lower 25% | 36 | 21 |
| carcinoma tissue | median | 130 | 101 |
| (25 samples) | upper 75% | 337 | 971 |
| Kidney chronic | lower 25% | 20 | 13 |
| inflammation tissue | median | 31 | 27 |
| (9 samples) | upper 75% | 46 | 63 |
| Normal kidney | lower 25% | −1 | 63 |
| tissue adjacent to | median | 22 | 86 |
| benign kidney tissue | upper 75% | 32 | 100 |
| (6 samples) | | | |
| Kidney | lower 25% | 31 | 19 |
| glomerulosclerosis | median | 37 | 31 |
| tissue (6 samples) | upper 75% | 51 | 38 |
| Kidney Wilms | lower 25% | 13 | 0 |
| tumor tissue | median | 27 | 11 |
| (6 samples) | upper 75% | 34 | 16 |
| Kidney | Expression | CD70 | CD203c |
| Tissue Type | Value Type | Expression | Expression |
| Kidney transitional | lower 25% | 56 | −16 |
| cell carcinoma | median | 65 | −15 |
| tissue (3 samples) | upper 75% | 70 | −3 |
| Kidney oncocytoma | lower 25% | 18 | 1 |
| tissue (5 samples) | median | 26 | 8 |
| | upper 75% | 31 | 11 |
| Kidney cyst tissue | lower 25% | 40 | 13 |
| (3 samples) | median | 40 | 18 |
| | upper 75% | 41 | 22 |

The distribution of CD70 and CD203c expression found at FIGS. 3, 4 (for CD70) and FIGS. 7 and 8 for (CD203c) was obtained from an analysis of the BioExpress™ database by the following method.

First, in order to create the information subset for “normal” tissue samples for the data analysis set forth in FIGS. 3 and 7, we gathered information related to those tissues that were labeled as “normal” in the BioExpress™ database. This subset contained 1200 samples. A second subset of samples was created for the malignant tissues considered in FIGS. 4 and 8. This subset was created by selecting information from those tissues that were identified by one of the following keywords: “malignant,” “adenoma,” “blastoma,” “carcinoma,” “sarcoma,” and “leukemia.” Second, the qualifiers for each of CD70 (i.e., “34054_at”) and CD203c (i.e., “89860_at”) were considered for each sample in the normal and malignant tissue subsets. (A “qualifier” is the code that Affymetrix ascribes to the probe pair in a microarray that measures whether a gene is present.)

Not all tissues in the normal and malignant tissue subsets had data pertaining to CD70 and/or CD203c gene expression. This is because some of the tissues may not have been analyzed using all five of the arrays in the GeneChip® Human Genome U95 Set. Consequently, the size of each of the normal and malignant tissue subsets for CD70 and CD203c is different, and is set forth in Table 2. The numbers in parentheses denote, in each subset, the number of tissue samples that yielded present calls for CD70 or CD203c gene expression, as the case may be. In viewing the distribution results for CD70 and CD203c gene expression, it should be noted that a margin of error is always present in classifying tissues as normal or malignant. For example, some of the tissue samples labeled as “normal” may have been derived from the periphery of a biopsied malignant sample and so may, in fact, not be entirely normal.

TABLE 2

| | Normal Tissue | Malignant Tissue |
|---|---|---|
| | Subset (Pcalls) | Subset (Pcalls) |
| CD70 gene expression | 1285 (26 or 2%) | 641 (96 or 15%) |
| CD203c gene expression | 1222 (349 or 28.5%) | 625 (180 or 28.8%) |

As can be seen from Table 2, the percentage of present calls for CD70 was very low in normal tissues and higher in malignant tissues. This makes CD70 an attractive molecular vehicle to use when attempting to target the malignant cells of a kidney, particularly renal cell carcinoma and clear cell renal cell carcinoma cells, by a therapeutic.

In this regard, since the percentage of present calls in normal and malignant tissues is about the same for CD203c gene expression, CD203c initially appears to be a less effective molecular vehicle to use for therapy unless a therapeutic composition is delivered specifically to the malignant kidney tissue. However, as suggested in the earlier discussion, it is important not to place too much weight on present call data only, since the actual levels of gene expression (i.e., average difference values) can be significantly different among samples exhibiting present call values. This, for example, is the case with CD203c, as is apparent from FIGS. 7 and 8. In fact, as discussed, FIGS. 7 and 8 suggest that CD203c gene expression in malignant kidney tissue is substantially greater than in most other malignant and normal tissues. This can be seen by looking, in particular, at the level of the bars in FIGS. 7 and 8 which, as noted earlier, depict median average difference values. Consequently, CD203c also can be exploited as a molecular vehicle that assists in targeting renal cell carcinoma and clear cell renal cell carcinoma cells by a therapeutic.

Moreover, the abnormally high amounts of CD70 and CD203c in malignant kidney tissue, particularly clear cell renal cell carcinoma and renal cell carcinoma, are an indication of disease status, and so forms the basis for diagnostic applications. For example, it becomes possible to use CD70 and CD203c as markers for kidney cancer, and particularly for renal cell carcinoma and clear cell renal cell carcinoma. Preferably, a cell-surface molecule can act as a marker when it is seen in negligible amounts in normal cells or not at all in such cells, and only at high levels in ill-functioning cells. While it is not so much of a problem if the molecule is present in malignant cells (other than those being targeted), CD70 and CD203c expression is generally also found to be higher in renal cell carcinoma and clear cell renal cell carcinoma than in other malignant and normal tissue.

Accordingly, kidney cancer, particularly renal cell carcinoma and clear cell renal cell carcinoma, could be diagnosed in a mammalian patient, preferably a human patient, by measuring the levels of CD70 and/or CD203c in a patient sample. For example, the amount of CD70 and/or CD203c in a patient sample can be determined by measuring the level of CD70 and/or CD203c gene expression in the sample. Although CD70 and/or CD203c levels could be measured in a patient's body tissue sample, such as a kidney tissue sample, we also believe that the levels of the soluble forms of each of CD70 and CD203c could be measured in a patient's body fluid sample, such as a blood sample, in order to diagnose kidney cancer, and specifically renal cell carcinoma and clear cell renal cell carcinoma. That is, an elevated amount of CD70 and/or CD203c in a patient sample compared to a suitable normal body fluid or tissue sample acting as a control suggests that the patient has kidney cancer.

We also envision that kidney carcinoma, particularly renal cell carcinoma or clear cell renal cell carcinoma, could be diagnosed in a mammalian (e.g., a human) patient by: conducting a database analysis of CD70 expression in human tissues (e.g., normal and/or malignant tissue) and preparing a profile of such expression; taking a sample of kidney tissue from the patient; detecting the amount of CD70 expression that is present in the patient sample; and comparing the amount of CD70 expression in the patient sample with the amounts of CD70 expression shown in the profile, wherein an amount of CD70 expression in the patient sample that is higher than the amounts of CD70 expression shown in the profile suggests that the patient is suffering from kidney carcinoma.

Similarly, we envision that kidney carcinoma, particularly renal cell carcinoma or clear cell renal cell carcinoma, could be diagnosed in a mammalian (e.g., a human) patient by: conducting a database analysis of CD203c expression in human tissues (e.g., normal and/or malignant tissue) and preparing a profile of such expression; taking a sample of kidney tissue from the patient; detecting the amount of CD203c expression that is present in the patient sample; and comparing the amount of CD203c expression in the patient sample with the amounts of CD203c expression shown in the profile, wherein an amount of CD203c expression in the patient sample that is higher than the amounts of CD203c expression shown in the profile suggests that the patient is suffering from kidney carcinoma.

Moreover, both CD70 and CD203c could be measured in a mammalian (e.g., a human) patient sample to render the diagnosis of kidney cancer (and specifically, renal cell carcinoma or clear cell renal cell carcinoma) more accurate. For example, such an assay could involve: taking a kidney tissue sample or a blood sample from a patient; detecting the amounts of both CD70 and CD203c that are present in the patient sample; and comparing the amounts of CD70 and CD203c in the patient sample as against the amounts of CD70 and CD203c found in a suitable, normal kidney tissue sample or blood sample acting as a control, wherein amounts of CD70 and/or CD203c in the patient sample that are higher than normal suggest that the patient is suffering from renal cell carcinoma or clear cell renal cell carcinoma. Finally, a diagnostic kit could be prepared for detecting kidney carcinoma in a sample of kidney tissue or body fluid that contains a reagent that is capable of detecting the presence of an elevated amount of CD70 in the sample, and a reagent that is capable of detecting the presence of CD203c in the sample.

Furthermore, as suggested earlier, CD70 and CD203c can be used as therapeutic targets to treat kidney cancer, particularly renal cell carcinoma and clear cell renal cell carcinoma. All potential therapeutic applications are based on the property of cells displaying different amounts (including presence and absence differences) of CD70 and CD203c. For example, these cell surface molecules are present in relatively large amounts in malignant kidney tissue as compared to other malignant human tissues, and in large amounts in renal cell carcinoma and clear cell renal cell carcinoma tissue, compared to in other normal and malignant tissues.

Typical applications for this invention include using antibodies or other molecules that specifically target CD70 and/or CD203c to deliver molecularly linked cellular killing agents, including cytotoxic and/or radioactive agents, specifically into diseased cells. For example, a pharmaceutical composition could be prepared containing: a hybrid molecular structure that includes a molecule that specifically targets CD70 or CD203c linked to a cellular killing agent; and a pharmaceutically acceptable carrier. We envision that the growth of a kidney tumor, particularly a renal cell carcinoma or a clear cell renal cell carcinoma tumor could be inhibited by administering to the tumor a pharmaceutically effective amount of such a hybrid molecular structure. For example, Ki-24 is a known anti-CD70 monoclonal antibody, and 97A6 is a known anti-CD203c monoclonal antibody, each or both of which could be used together with a cellular killing agent to create an immunoconjugate that could be used to treat a patient that has or is at risk of developing renal cell carcinoma or clear cell renal cell carcinoma. In addition, CD27 specifically targets CD70 and could also be used in such a hybrid molecular structure. Also, enediyene antitumor antibiotics, such as the calicheamicins as well as calicheamicin derivatives, can be used as a cytotoxic agent that can be linked to the antibody or other molecule with high affinity to either CD70 or CD203c or both. The potent family of antibacterial and antitumor agents known collectively as the calicheamicins (or the LL-E33288 complex), are described and claimed in U.S. Pat. No. 4,970,198, the content of which is herein incorporated by reference in its entirety. Moreover, the N-acylated derivatives and disulfide analogs, for example, of such calicheamicins are described, respectively, in U.S. Pat. Nos. 5,079,233 and 5,606,040, the content of each of which also is herein incorporated by reference in its entirety.

The strategy of creating an immunoconjugate by linking a cytotoxic agent, such as a calicheamicin or a calicheamicin derivative, to an antibody or other molecule which targets an undesired population of cells has been used successfully, for example, to create Mylotarg™. In the case of Mylotarg™, an anti-CD33 antibody is linked by way of a chemical linker to a calicheamicin derivative. This drug is currently indicated for use in the treatment of CD33 positive relapsed acute myelogenous leukemia (AML). The immunoconjugate technology used in Mylotarg™ and which is equally applicable here is described extensively in U.S. Pat. Nos. 5,739,116, 5,767,285 and 5,773,001, the content of each of which is herein incorporated by reference in its entirety. An immunoconjugate to treat kidney cancer, such as renal cell carcinoma and clear cell renal cell carcinoma, as described previously, could be administered, in a pharmaceutically effective dose, either directly to a patient's malignant kidney tissue, or indirectly to that tissue by administering such a conjugate to the patient by any known method, including orally and parenterally. Indirect administration could be beneficial in treating metastatic forms of renal cell carcinoma and clear cell renal cell carcinoma. More details about the preparation and use of such an immunoconjugate are set forth in the Example provided below.

Furthermore, an antibody to CD70 and/or CD203c could itself be used to induce cell death. In addition, a peptide fragment or a synthetic compound that exhibits affinity to these molecules also can be used on its own to deliver agents into the diseased cells, or to cause a direct kill or modification to the diseased cells. Any such compositions also could be administered, in a pharmaceutically effective dose, either directly to a patient's malignant kidney tissue, or indirectly to that tissue by administering the composition to the patient by any known method, including orally and parenterally. As suggested above, indirect administration of such compositions could be beneficial in treating metastatic forms of renal cell carcinoma and clear cell renal cell carcinoma.

We envision that one could reduce or eliminate kidney tumor growth, particularly renal cell carcinoma tissue and clear cell renal cell carcinoma tissue growth, by reducing the amount of CD70 and/or CD203c molecules in a patient, for example by reducing the amounts of these molecules or of CD70 and CD203c gene expression in the patient's circulating blood and/or in the tumor tissue itself. For example, reducing CD70 and/or CD203c gene expression could be done using a strategy that involves RNA interference (RNAi) techniques, as described generally in U.S. Pat. No. 6,506,559 B1 and U.S. Pat. No. 6,573,099 B2. See also, for example, Scherr, M. et al., “Gene Silencing Mediated by Small Interfering RNAs in Mammalian Cells,” Current Medicinal Chemistry 10: 245-256 (2003).

Essentially, RNAi, also referred to as Post-Transcriptional Gene Silencing (PTGS), is a biological mechanism that is initiated by double-stranded RNA (dsRNA) and mediates the degradation of homologous mRNA in eukaryotic cells. Double-stranded RNA (dsRNA) is processed into small interfering RNAs (siRNA) which are about 21 nucleotides in length with 3′-overhangs. It is these siRNAs that mediate sequence-specific mRNA degradation. In mammalian cells, siRNAs can be used as mediators of sequence-specific mRNA degradation so as to avoid the non-specific gene silencing that is induced by longer dsRNA. Preferably, it appears that the siRNAs should be less than about 30 base pairs to avoid the non-specific mRNA degradation pathway in mammalian cells.

We believe that siRNAs could be generated that target complementary RNA molecules coding for either CD70 or CD203c, and then cleave and destroy such RNA, so as to inhibit CD70 and/or CD203c gene expression. Suitable siRNAs can be exogenously delivered to the target cells containing CD70 and/or CD203c expression sought to be inhibited, or can be endogenously expressed from appropriate expression cassettes in such target cells. If delivered exogenously, the siRNAs can be chemically or in vitro enzymatically synthesized. Most preferably, the target cells in which CD70 and/or CD203c expression is sought to be inhibited are renal cell carcinoma cells and clear cell renal cell carcinoma cells.

Preferably, suitable siRNAs for targeting CD70 or CD203c expression are selected such that they are complementary to a portion of the mRNA sequence of either CD70 or CD203c (i.e., a target site) based on the following rules.

(1) The target site preferably should be 21 nucleotides in length (i.e., a “21-mer”) and preferably should start with one of the following two base pairs: AA, UA, GA or CA. Most preferably, the first two base pairs in the 21-mer are AA.

(2) The GC % content in the target site preferably should be between about 45% and about 55%.

(3) Preferably, in the target site, there should not be about 3 or more of the same base pair contiguously, in a row. That is, most preferably there should be no GGG, CCC, UUU or AAA.

(4) Preferably, there should not be about seven or more contiguous G/Cs (in any order) in a row in the target site.

(5) Preferably, target sites should only be found in the open reading frame (ORF) region, about 75 base pairs after the starting AUG and about 75 base pairs before the stopping codon.

Given these rules for preferred siRNA target site picking, the following tables, i.e., Tables 3 and 4, provide prediction results for suitable siRNA target sites in each of CD70 and CD203c mRNA sequences, respectively.

The complete mRNA sequences for each of CD70 and CD203c from which the siRNA target site predictions provided in Tables 3 and 4 are derived are set forth, respectively, as SEQ ID NO: 1 and SEQ ID NO: 2, and are also publicly available in the National Center for Biotechnology Information (NCBI) nucleotide database under record numbers NM_{—}001252 and NM_{—}005021, respectively, albeit in a DNA form. The first column in each of Tables 3 and 4 provides the segment of mRNA in either CD70 or CD203c that would be targeted by the siRNA strand provided in the fourth and fifth columns that is in the same row. For example, SEQ ID NO: 3 is the target segment for the predicted siRNA composed of SEQ ID NO: 17 as the sense strand and SEQ ID NO: 31 as the antisense strand. In addition, each of these tables provides information as to the “GC” content of the target segment in the second column, and the target segment's position location within the complete mRNA in the third column.

We envision that the predicted siRNAs in Table 3 and Table 4 could be used, as a therapeutic strategy, to inhibit, respectively, CD70 or CD203c expression in a patient. In particular, such predicted siRNAs could be used to inhibit CD70 or CD203c expression in kidney carcinoma cells, particularly in renal cell carcinoma and clear cell renal cell carcinoma cells, so as to reduce the growth of tumors containing such cells. A pharmaceutically effective amount of at least one of the predicted siRNAs could be delivered exogenously to the cells in the malignant kidney tissue. Such exogenous delivery could be by a direct infusion of the siRNA to the malignant kidney tissue. However, such an siRNA also can be administered to a patient by any other known delivery method, such as orally or parenterally. Oral and parenteral administration could be helpful to treat metastatic kidney cancer. Alternatively, such an siRNA can be endogenously expressed in the patient's body, for example, in the malignant kidney tissue. Each such siRNA could, for example, contain a sense strand (5′→3′) together with its complementary siRNA antisense strand (3′→5′).

TABLE 3

siRNA Target Site Prediction Results for CD70 Coding Sequence

| | | | | |
|---|---|---|---|---|
| | | | siRNA Sense | siRNA Antisense |
| Target segment: 5′→3′ | GC Content | Position | strand: 5′→3′ | strand: 3′→5′ |

| | | | | |
|---|---|---|---|---|
| Target segment starts | | | | |
| with AA | | | | |
| AAUCACACAGGACCUCAGCAG | 0.52 | 186 | UCACACAGGACCUCAGCAGUU | UUAGUGUGUCCUGGAGUCGUC |
| (SEQ ID NO: 3) | | | (SEQ ID NO: 17) | (SEQ ID NO: 31) |
| Target segment starts | | | | |
| with CA | | | | |
| CAGCUGAAUCACACAGGACCU | 0.52 | 180 | GCUGAAUCACACAGGACCUUU | UUCGACUUAGUGUGUCCUGGA |
| (SEQ ID NO: 4) | | | (SEQ ID NO: 18) | (SEQ ID NO: 32) |
| CAGCUACGUAUCCAUCGUGAU | 0.48 | 282 | GCUACGUAUCCAUCGUGAUUU | UUCGAUGCAUAGGUAGCACUA |
| (SEQ ID NO: 5) | | | (SEQ ID NO: 19) | (SEQ ID NO: 33) |
| CAUCGUGAUGGCAUCUACAUG | 0.48 | 294 | UCGUGAUGGCAUCUACAUGUU | UUAGCACUACCGUAGAUGUAC |
| (SEQ ID NO: 6) | | | (SEQ ID NO: 20) | (SEQ ID NO: 34) |
| CAUGGUACACAUCCAGGUGAC | 0.52 | 311 | UGGUACACAUCCAGGUGACUU | UUACCAUGUGUAGGUCCACUG |
| (SEQ ID NO: 7) | | | (SEQ ID NO: 21) | (SEQ ID NO: 35) |
| CAGCUUCCACCAAGGUUGUAC | 0.52 | 434 | GCUUCCACCAAGGUUGUACUU | UUCGAAGGUGGUUCCAACAUG |
| (SEQ ID NO: 8) | | | (SEQ ID NO: 22) | (SEQ ID NO: 36) |
| CACCAAGGUUGUACCAUUGCC | 0.52 | 441 | CCAAGGUUGUACCAUUGCCUU | UUGGUUCCAACAUGGUAACGG |
| (SEQ ID NO: 9) | | | (SEQ ID NO: 23) | (SEQ ID NO: 37) |
| CAAGGUUGUACCAUUGCCUCC | 0.52 | 444 | AGGUUGUACCAUUGCCUCCUU | UUUCCAACAUGGUAACGGAGG |
| (SEQ ID NO: 10) | | | (SEQ ID NO: 24) | (SEQ ID NO: 38) |
| Target segment starts | | | | |
| with GA | | | | |
| GAGCUGCAGCUGAAUCACACA | 0.52 | 174 | GCUGCAGCUGAAUCACACAUU | UUCGACGUCGACUUAGUGUGU |
| (SEQ ID NO: 11) | | | (SEQ ID NO: 25) | (SEQ ID NO: 39) |
| GAAUCACACAGGACCUCAGCA | 0.52 | 185 | AUCACACAGGACCUCAGCAUU | UUUAGUGUGUCCUGGAGUCGU |
| (SEQ ID NO: 12) | | | (SEQ ID NO: 26) | (SEQ ID NO: 40) |
| GAUGGCAUCUACAUGGUACAC | 0.48 | 300 | UGGCAUCUACAUGGUACACUU | UUACCGUAGAUGUACCAUGUG |
| (SEQ ID NO: 13) | | | (SEQ ID NO: 27) | (SEQ ID NO: 41) |
| Target segment starts | | | | |
| with UA | | | | |
| UAGCUGAGCUGCAGCUGAAUC | 0.52 | 169 | GCUGAGCUGCAGCUGAAUCUU | UUCGACUCGACGUCGACUUAG |
| (SEQ ID NO: 14) | | | (SEQ ID NO: 28) | (SEQ ID NO: 42) |
| UACGUAUCCAUCGUGAUGGCA | 0.48 | 286 | CGUAUCCAUCGUGAUGGCAUU | UUGCAUAGGUAGCACUACCGU |
| (SEQ ID NO: 15) | | | (SEQ ID NO: 29) | (SEQ ID NO: 43) |
| UACAUGGUACACAUCCAGGUG | 0.48 | 309 | CAUGGUACACAUCCAGGUGUU | UUGUACCAUGUGUAGGUCCAC |
| (SEQ ID NO: 16) | | | (SEQ ID NO: 30) | (SEQ ID NO: 44) |

TABLE 4

siRNA Target Site Prediction Results for CD203c Coding Sequence

| | | | | |
|---|---|---|---|---|
| | | | siRNA Sense | siRNA Antisense |
| Target segment: 5′→3′ | GC Content | Position | strand: 5′→3′ | strand: 3′→5′ |

| | | | | |
|---|---|---|---|---|
| Target segment starts | | | | |
| with AA | | | | |
| AAGGCAGCUGCAGGAAGAAGU | 0.52 | 168 | GGCAGCUGCAGGAAGAAGUUU | UUCCGUCGACGUCCUUCUUCA |
| (SEQ ID NO: 45) | | | (SEQ ID NO: 91) | (SEQ ID NO: 137) |
| AAGACCGAGGUGAUUGCUGCU | 0.52 | 243 | GACCGAGGUGAUUGCUGGUUU | UUGUGGCUCCACUAACGACGA |
| (SEQ ID NO: 46) | | | (SEQ ID NO: 92) | (SEQ ID NO: 138) |
| AAUAAUCCAGCCUGGUGGCAU | 0.48 | 749 | UAAUCCAGCCUGGUGGCAUUU | UUAUUAGGUCGGACCACCGUA |
| (SEQ ID NO: 47) | | | (SEQ ID NO: 93) | (SEQ ID NO: 139) |
| AACCAAUGUGGCUGACAGCAA | 0.48 | 774 | CCAAUGUGGCUGACAGCAAUU | UUGGUUACACCGACUGUCGUU |
| (SEQ ID NO: 48) | | | (SEQ ID NO: 94) | (SEQ ID NO: 140) |
| AAGAACCUGAUUCCUCUGGAC | 0.48 | 981 | GAACCUGAUUCCUCUGGACUU | UUCUUGGACUAAGGAGACCUG |
| (SEQ ID NO: 49) | | | (SEQ ID NO: 95) | (SEQ ID NO: 141) |
| AACCUGAUUCCUCUGGACAUG | 0.48 | 984 | CCUGAUUCCUCUGGACAUGUU | UUGGACUAAGGAGACCUGUAC |
| (SEQ ID NO: 50) | | | (SEQ ID NO: 96) | (SEQ ID NO: 142) |
| AAGGCCUGAAGCAGCGGAAUU | 0.52 | 1077 | GGCCUGAAGCAGCGGAAUUUU | UUCCGGACUUCGUCGCCUUAA |
| (SEQ ID NO: 51) | | | (SEQ ID NO: 97) | (SEQ ID NO: 143) |
| AAGCGACUGCACUAUGCCAAG | 0.52 | 1352 | GCGACUGCACUAUGCCAAGUU | UUCGCUGACGUGAUACGGUUC |
| (SEQ ID NO: 52) | | | (SEQ ID NO: 98) | (SEQ ID NO: 144) |
| AACAGUGGCUGGCUGUUAGGA | 0.52 | 1410 | CAGUGGCUGGCUGUUAGGAUU | UUGUCACCGACCGACAAUCCU |
| (SEQ ID NO: 53) | | | (SEQ ID NO: 99) | (SEQ ID NO: 145) |
| AAUUGUGGAGGAGGCAACCAU | 0.48 | 1445 | UUGUGGAGGAGGCAACCAUUU | UUAACACCUCCUCCGUUGGUA |
| (SEQ ID NO: 54) | | | (SEQ ID NO: 100) | (SEQ ID NO: 146) |
| AACCAUCUUCUGAAGGUGCCU | 0.48 | 1634 | CCAUCUUCUGAAGGUGCCUUU | UUGGUAGAAGACUUCCACGGA |
| (SEQ ID NO: 55) | | | (SEQ ID NO: 101) | (SEQ ID NO: 147) |
| AAGAACGUGGACCACUGUCUC | 0.52 | 1868 | GAACGUGGACCACUGUCUCUU | UUCUUGGACCUGGUGACAGAG |
| (SEQ ID NO: 56) | | | (SEQ ID NO: 102) | (SEQ ID NO: 148) |
| AACGUGGACCACUGUCUCCUU | 0.52 | 1871 | CGUGGACCACUGUCUCCUUUU | UUGCACCUGGUGACAGAGGAA |
| (SEQ ID NO: 57) | | | (SEQ ID NO: 103) | (SEQ ID NO: 149) |
| AACAAGAGCCACACACCGGAA | 0.52 | 2381 | CAAGAGCCACACACCGGAAUU | UUGUUCUCGGUGUGUGGGCUU |
| (SEQ ID NO: 58) | | | (SEQ ID NO: 104) | (SEQ ID NO: 150) |
| AACGUGGAGAGCUGUCCUGAA | 0.52 | 2456 | CGUGGAGAGCUGUCCUGAAUU | UUGCACCUCUCGACAGGACUU |
| (SEQ ID NO: 59) | | | (SEQ ID NO: 105) | (SEQ ID NO: 151) |
| Target segment starts | | | | |
| with CA | | | | |
| CAUGUCACUUGGAUUAGGCCU | 0.48 | 118 | UGUCACUUGGAUUAGGCCUUU | UUACAGUGAACCUAAUCCGGA |
| (SEQ ID NO: 60) | | | (SEQ ID NO: 106) | (SEQ ID NO: 152) |
| CAGCUGCAGGAAGAAGUGCUU | 0.52 | 172 | GCUGCAGGAAGAAGUGCUUUU | UUCGACGUCCUUCUUCACGAA |
| (SEQ ID NO: 61) | | | (SEQ ID NO: 107) | (SEQ ID NO: 153) |
| CACCUGUGUGGAAUCAACUCG | 0.52 | 277 | CCUGUGUGGAAUCAACUCGUU | UUGGACACACCUUAGUUGAGC |
| (SEQ ID NO: 62) | | | (SEQ ID NO: 108) | (SEQ ID NO: 154) |
| CAGAGUCACAUGGCAUCAUUG | 0.48 | 669 | GAGUCACAUGGCAUCAUUGUU | UUCUCAGUGUACCGUAGUAAC |
| (SEQ ID NO: 63) | | | SEQ ID NO: 109) | (SEQ ID NO: 155) |
| CAACCAAUGUGGCUGACAGCA | 0.52 | 773 | ACCAAUGUGGCUGACAGCAUU | UUUGGUUACACCGACUGUCGU |
| (SEQ ID NO: 64) | | | (SEQ ID NO: 110) | (SEQ ID NO: 156) |
| CAAUGUGGCUGACAGCAAUGU | 0.48 | 777 | AUGUGGCUGACAGCAAUGUUU | UUUACACCGACUGUCGUUACA |
| (SEQ ID NO: 65) | | | (SEQ ID NO: 111) | (SEQ ID NO: 157) |
| CAUGCCUUACAACGGAAGUGU | 0.48 | 874 | UGCCUUACAACGGAAGUGUUU | UUACGGAAUGUUGCCUUCACA |
| (SEQ ID NO: 66) | | | (SEQ ID NO: 112) | (SEQ ID NO: 158) |
| CACUAUGCCAAGAACGUCAGA | 0.48 | 1361 | CUAUGCCAAGAACGUCAGAUU | UUGAUACGGUUCUUGCAGUCU |
| (SEQ ID NO: 67) | | | (SEQ ID NO: 113) | (SEQ ID NO: 159) |
| CAGCUGGAACAAGUGAAUCAG | 0.48 | 1769 | GCUGGAACAAGUGAAUCAGUU | UUCGACCUUGUUCACUUAGUC |
| (SEQ ID NO: 68) | | | (SEQ ID NO: 114) | (SEQ ID NO: 160) |
| CAGAAGAACGUGGACCAGUGU | 0.52 | 1865 | GAAGAACGUGGACCACUGUUU | UUCUUCUUGCACCUGGUGACA |
| (SEQ ID NO: 69) | | | (SEQ ID NO: 115) | (SEQ ID NO: 161) |
| Target segment starts | | | | |
| with GA | | | | |
| GAAGACACCUGUGUGGAAUCA | 0.48 | 272 | AGACACCUGUGUGGAAUCAUU | UUUCUGUGGACACACCUUAGU |
| (SEQ ID NO: 70) | | | (SEQ ID NO: 116) | (SEQ ID NO: 162) |
| GACACCUGUGUGGAAUCAACU | 0.48 | 275 | CACCUGUGUGGAAUCAACUUU | UUGUGGACACACCUUAGUUGA |
| (SEQ ID NO: 71) | | | (SEQ ID NO: 117) | (SEQ ID NO: 163) |
| GAGAGACCAGAUUAGAGGCCA | 0.52 | 327 | GAGACCAGAUUAGAGGCCAUU | UUCUCUGGUCUAAUCUCCGGU |
| (SEQ ID NO: 72) | | | (SEQ ID NO: 118) | (SEQ ID NO: 164) |
| GACCUGCCACCAGUUAUCUUG | 0.52 | 485 | CCUGCCACCAGUUAUCUUGUU | UUGGACGGUGGUCAAUAGAAC |
| (SEQ ID NO: 73) | | | (SEQ ID NO: 119) | (SEQ ID NO: 165) |
| GAGUCACAUGGCAUCAUUGAC | 0.48 | 671 | GUCACAUGGCAUCAUUGACUU | UUCAGUGUACCGUAGUAACUG |
| (SEQ ID NO: 74) | | | (SEQ ID NO: 120) | (SEQ ID NO: 166) |
| GAAGAAGCUGAUUCCUCUGGA | 0.48 | 980 | AGAACCUGAUUCCUCUGGAUU | UUUCUUGGACUAAGGAGACCU |
| (SEQ ID NO: 75) | | | (SEQ ID NO: 121) | (SEQ ID NO: 167) |
| GAACCUGAUUCCUCUGGACAU | 0.48 | 983 | ACCUGAUUCCUCUGGACAUUU | UUUGGACUAAGGAGACCUGUA |
| (SEQ ID NO: 76) | | | (SEQ ID NO: 122) | (SEQ ID NO: 168) |
| GAUUCCUCUGGACAUGCAGGU | 0.52 | 989 | UUCCUCUGGACAUGCAGGUUU | UUAAGGAGACCUGUACGUCCA |
| (SEQ ID NO: 77) | | | (SEQ ID NO: 123) | (SEQ ID NO: 169) |
| GACCAGUCAGUGCCAGAGUAA | 0.52 | 1011 | CCAGUCAGUGCCAGAGUAAUU | UUGGUCAGUCACGGUCUCAUU |
| (SEQ ID NO: 78) | | | (SEQ ID NO: 124) | (SEQ ID NO: 170) |
| GAUGUUGAUGGAAGGCCUGAA | 0.48 | 1066 | UGUUGAUGGAAGGCCUGAAUU | UUACAACUACCUUCCGGACUU |
| (SEQ ID NO: 79) | | | (SEQ ID NO: 125) | (SEQ ID NO: 171) |
| GACCAUGGAAUGGACCAGACU | 0.52 | 1130 | CCAUGGAAUGGACCAGACUUU | UUGGUACCUUAGCUGGUCUGA |
| (SEQ ID NO: 80) | | | (SEQ ID NO: 126) | (SEQ ID NO: 172) |
| GACUGCACUAUGCCAAGAACG | 0.52 | 1356 | CUGCACUAUGCCAAGAACGUU | UUGACGUGAUACGGUUCUUGC |
| (SEQ ID NO: 81) | | | (SEQ ID NO: 127) | (SEQ ID NO: 173) |
| GAUCAACAGUGGCUGGCUGUU | 0.52 | 1406 | UCAACAGUGGCUGGCUGUUUU | UUAGUUGUCACCGACCGACAA |
| (SEQ ID NO: 82) | | | (SEQ ID NO: 128) | (SEQ ID NO: 174) |
| GAGGAGGCAACCAUGGUUAUA | 0.48 | 1452 | GGAGGCAACCAUGGUUAUAUU | UUCCUCCGUUGGUACCAAUAU |
| (SEQ ID NO: 83) | | | (SEQ ID NO: 129) | (SEQ ID NO: 175) |
| GAAGAACGUGGACCACUGUCU | 0.52 | 1867 | AGAACGUGGACCACUGUCUUU | UUUCUUGCACCUGGUGACAGA |
| (SEQ ID NO: 84) | | | (SEQ ID NO: 130) | (SEQ ID NO: 176) |
| Target segment starts | | | | |
| with UA | | | | |
| UAUCCAGAGUCACAUGGCAUC | 0.48 | 665 | UGCAGAGUCACAUGGCAUCUU | UUAGGUGUCAGUGUACCGUAG |
| (SEQ ID NO: 85) | | | (SEQ ID NO: 131) | (SEQ ID NO: 177) |
| UAUCAUGCUUCUGGCUGACCA | 0.48 | 1114 | UCAUCCUUCUGGCUGACCAUU | UUAGUAGGAAGACCGACUGGU |
| (SEQ ID NO: 86) | | | (SEQ ID NO: 132) | (SEQ ID NO: 178) |
| UAGGAGCAUGGAGGCUAUCUU | 0.48 | 1483 | GGAGCAUGGAGGCUAUCUUUU | UUCCUCGUACCUCCGAUAGAA |
| (SEQ ID NO: 87) | | | (SEQ ID NO: 133) | (SEQ ID NO: 179) |
| UACGCAUUCAACCAGCACCAA | 0.48 | 1590 | CGCAUUCAACCAGCACCAAUU | UUGGGUAAGUUGGUCGUGGUU |
| (SEQ ID NO: 88) | | | (SEQ ID NO: 134) | (SEQ ID NO: 180) |
| UACUGCAGAAGAACGUGGACC | 0.52 | 1860 | CUGCAGAAGAACGUGGACCUU | UUGACGUCUUCUUGCACCUGG |
| (SEQ ID NO: 89) | | | (SEQ ID NO: 135) | (SEQ ID NO: 181) |
| UAUCCUCCUGCCAGCAAUAGA | 0.48 | 2096 | UCCUCGUGCCAGCAAUAGAUU | UUAGGAGGACGGUCGUUAUCU |
| (SEQ ID NO: 90) | | | (SEQ ID NO: 136) | (SEQ ID NO: 182) |

The following Example is merely illustrative of one aspect of our invention and is not to be considered as limiting the invention, which is properly delineated in the following claims.

### EXAMPLE

We envision that an immunoconjugate for treating kidney cancer, specifically renal cell carcinoma or clear cell renal cell carcinoma, can be prepared and used as described in this Example.

1. Preparation of an Immunoconjugate that Targets CD70

An immunoconjugate that destroys malignant kidney cells, particularly renal cell carcinoma and clear cell renal cell carcinoma cells by targeting CD70 can be produced by linking a Ki-24 monoclonal antibody (MAb) (i.e., an anti-CD-70 monoclonal antibody) to a calicheamicin derivative.

The anti-CD70 Ki-24 antibody can be humanized if necessary (as described, for example, in U.S. Pat. Nos. 5,585,089 and 5,693,762, the content of each of which is herein incorporated by reference in its entirety) and then produced by mammalian cell suspension culture using a myeloma NSO cell line. The antibody may then be purified under conditions that remove or inactivate viruses. Three separate and independent steps can be used in this antibody purification process. These include: low pH treatment, DEAE-Sepharose chromatography, and viral filtration. Next, the anti-CD-70 Ki-24 antibody may be linked via a bifunctional linker to N-acetyl-gamma calicheamicin. The technology to prepare this immunoconjugate (including the linker technology), can be found in U.S. Pat. Nos. 4,970,198, 5,079,233, 5,606,040, 5,739,116, 5,767,285 and 5,773,001, the content of each of which is herein incorporated by reference in its entirety. The immunoconjugate can be prepared in which approximately 50% of anti-CD70 Ki-24 antibody is loaded with about 4-6 moles of calicheamicin per mole of antibody. The remaining approximately 50% of the antibody would not be linked to the calicheamicin derivative.

2. Preparation of an Immunoconjugate that Targets CD203c

A second immunoconjugate can be prepared as described in part 1 of this Example using a different monoclonal antibody, namely, 97A6, which is an anti-CD203c antibody, and linking it to a calicheamicin derivative, such as N-acetyl-gamma calicheamicin. This immunoconjugate would be expected to destroy malignant kidney cells, particularly renal cell carcinoma and clear cell renal carcinoma cells by targeting CD203c.

3. Mechanism of Action

We envision that the immunoconjugate prepared either in part 1 or part 2 of this Example would be primarily directed against either the CD70 or the CD203c antigen (as the case may be) expressed by renal cell carcinoma and clear cell renal cell carcinoma cells. We anticipate that binding of the anti-CD70 or the anti-CD203c antibody portion of the immunoconjugate with either the CD70 or the CD203c antigen will result in the formation of a complex that will end up getting internalized by the malignant cells. Upon internalization, we anticipate that the calicheamicin derivative will be released inside lysosomes of the malignant cells. The released calicheamicin derivative inside the malignant cells could then bind to DNA of the malignant cell in the minor groove which would result in DNA double strand breaks and malignant cell death.

4. Preclinical Studies

We envision that an immunoconjugate as described in part 1 of this Example would be cytotoxic to a CD70 positive malignant kidney cell line, if such an immunoconjugate were appropriately delivered thereto. Similarly, we envision that an immunoconjugate, as described in part 2 of this Example, would be cytotoxic to a CD203c-positive malignant kidney cell line, if such an immunoconjugate were appropriately delivered thereto. Moreover, we believe the immunoconjugates described in parts 1 and 2 also would exhibit antitumor effects in suitable animal models that contain CD70 positive and/or CD203c positive cell lines, if such immunoconugates were to be appropriately administered (i.e., either directly to the malignant kidney tissue in the animal or indirectly thereto by an oral or parenteral route known by those skilled in the art).

5. Human Administration

We envision that a pharmaceutically effective amount of the immunoconjugate described in parts 1 or 2 of this Example can be administered to a human patient suffering from kidney cancer, specifically renal cell carcinoma or clear cell renal cell carcinoma, by methods known to those skilled in the art. For example, an appropriate dose and method of administration of the immunoconjugate can be a dose of 9 mg/m², administered as a 2-hour intravenous infusion. An appropriate treatment course can be, for example, 2 doses with 14 days between the doses. The described treatment can occur on an outpatient basis.

While particular embodiments of the present invention have been illustrated and described, it will be apparent to those skilled in the art that various changes and modifications may be made without departing from the spirit and scope of the invention. Furthermore, it is intended that the claims will cover all such modifications that are within the scope of the invention.

1. A method of diagnosing kidney cancer in a mammalian patient comprising the steps of:
taking a sample of body fluid or tissue from the patient;
detecting the amount of CD70 that is present in the patient sample; and
comparing the amount of CD70 in the patient sample as against the amount of CD70 in a suitable, normal mammalian body fluid or tissue sample acting as a control,
wherein an elevated amount of CD70 in the patient sample compared to the control suggests that the patient has kidney cancer.

2. The method of claim 1, wherein the mammalian patient is a human patient, the kidney cancer is renal cell carcinoma or clear cell renal cell carcinoma, and the patient sample is selected from the group consisting of a blood sample and a kidney tissue sample.

3. A method of diagnosing kidney cancer in a mammalian patient comprising the steps of:
taking a sample of body fluid or tissue from the patient;
detecting the amount of CD203c that is present in the patient sample; and
comparing the amount of CD203c in the patient sample as against the amount of CD203c in a suitable, normal mammalian body fluid or tissue sample acting as a control,
wherein an elevated amount of CD203c in the patient sample compared to the control suggests that the patient has kidney cancer.

4. The method of claim 3, wherein the mammalian patient is a human patient, the kidney cancer is renal cell carcinoma or clear cell renal cell carcinoma, and the patient sample is selected from the group consisting of a blood sample and a kidney tissue sample.

5. An assay to detect the presence of renal cell carcinoma cells or clear cell renal cell carcinoma cells in a human patient comprising the steps of:
taking a kidney tissue sample or a blood sample from the patient;
detecting the amounts of CD70 and CD203c that are present in the patient sample; and
comparing the amounts of CD70 and CD203c in the patient sample as against the amounts of CD70 and CD203c found in a suitable normal kidney tissue sample or blood sample acting as a control,
wherein amounts of at least one of CD70 and CD203c in the patient sample that are higher than normal, as compared to the control, suggest that the patient is suffering from renal cell carcinoma or clear cell renal cell carcinoma.

6. A pharmaceutical composition comprising:
a hybrid molecular structure, itself comprising a molecule that specifically targets CD70 linked to a cellular killing agent; and
a pharmaceutically acceptable carrier,
wherein the composition destroys malignant kidney tissue.

7. The pharmaceutical composition of claim 6, wherein the malignant kidney tissue is renal cell carcinoma tissue or clear cell renal cell carcinoma tissue.

8. The pharmaceutical composition of claim 6, wherein the cellular killing agent is a calicheamicin or a calicheamicin derivative.

9. The pharmaceutical composition of claim 6, wherein the molecule that specifically targets CD70 is CD27 or Ki-24.

10. A pharmaceutical composition comprising:
a hybrid molecular structure itself comprising a molecule that specifically targets CD203c linked to a cellular killing agent; and
a pharmaceutically acceptable carrier,
wherein the composition destroys malignant kidney tissue.

11. The pharmaceutical composition of claim 10, wherein the malignant kidney tissue is renal cell carcinoma tissue or clear cell renal cell carcinoma tissue.

12. The pharmaceutical composition of claim 10, wherein the cellular killing agent is a calicheamicin or a calicheamicin derivative.

13. The pharmaceutical composition of claim 10, wherein the molecule that specifically targets CD203c is 97A6.

14. A method of treating a human patient that has or is at risk of developing renal cell carcinoma or clear cell renal cell carcinoma using a targeted drug delivery approach comprising:
preparing an immunoconjugate comprising a cellular killing agent linked to a monoclonal antibody directed against CD70 or CD203c; and
administering the immunoconjugate to the patient in a pharmaceutically effective dose.

15. The method of claim 14, wherein the cellular killing agent is a cytotoxic agent or a radioactive agent.

16. The method of claim 14, wherein the cellular killing agent is selected from the group consisting of a calicheamicin and a calicheamicin derivative, and the monoclonal antibody is Ki-24 or 97A6.

17. A method of inhibiting the growth of a renal cell carcinoma tumor or a clear cell renal cell carcinoma tumor comprising:
preparing a hybrid molecular structure, itself comprising a cellular killing agent linked to a molecule that specifically targets at least one of CD70 and CD203c; and
delivering to the tumor a pharmaceutically effective amount of the hybrid molecular structure.

18. The method of claim 17, wherein the cellular killing agent is a cytotoxic agent or a radioactive agent.

19. The method of claim 17, wherein the cellular killing agent is a calicheamicin or a calicheamicin derivative, and the molecule that specifically targets at least one of CD70 and CD203c is Ki-24, CD27 or 97A6.

20. A method of treating a human patient that has or is at risk of developing renal cell carcinoma or clear cell renal cell carcinoma comprising:
administering directly or indirectly to the patient's kidneys a pharmaceutically effective dose of a preparation selected from the group consisting of:
an antibody to at least one of CD70 and CD203c that is capable of inducing cell death;
an antibody to at least one of CD70 and CD203c that is linked to a cellular killing agent;
a peptide fragment that exhibits affinity for at least one of CD70 and CD203c and that is capable of inducing cell death; or
a synthetic composition that exhibits affinity for at least one of CD70 and CD203c and that is capable of inducing cell death.

21. A method of reducing or stopping the growth of malignant kidney tissue in a mammalian patient comprising:
reducing the levels of at least one of CD70 and CD 203c in the patient.

22. The method of claim 21, wherein the levels of CD70 or CD203c in the patient are reduced by reducing the amounts of soluble CD70 or CD203c in the patient's circulating blood.

23. The method of claim 21, wherein the levels of CD70 or CD203c are reduced by reducing CD70 or CD203c gene expression using an RNA interference strategy that comprises the step of exogenously delivering to the cells in the malignant kidney tissue or endogenously expressing in the cells of the malignant kidney tissue an effective amount of at least one siRNA, each such siRNA being selected from the group consisting of siRNAs as shown in Table 3 and Table 4, wherein each such siRNA comprises a sense strand (5′→3′) together with its complementary siRNA antisense strand (3′→5′).

24. The method of claim 23, wherein the malignant kidney tissue is renal cell carcinoma tissue or clear cell renal cell carcinoma tissue.

25. The method of claim 21, wherein the levels of CD70 or CD203c in the patient are reduced by administering an RNAi molecule.
